SEQUENCE LISTING (5 sequences)

SEQ ID NO: 1 - protein, 189 aa, Homo sapiens
MASKRALVILAKGAEEMETVIPVDVMRRAGIKVTVAGLAGKDPVQCSRDVVICPDASLEDAKKEGPYDVVVLPGGNLGAQNLSESAAVKEILKEQENRKGLIAAICAGPTALLAHEIGFGSKVTTHPLAKDKMMNGGHYTYSENRVEKDGLILTSRGPGTSFEFALAIVEALNGKEVAAQVKAPLVLKD

SEQ ID NO: 2 - RNA, 1069 nt, Homo sapiens
  source 1..1069: mol_type = mRNA
gcgttcattttcagcctggtgtggggcttgtaaacatataacataaaaatggcttccaaaagagctctggtcatcctggctaaaggagcagaggaaatggagacggtcatccctgtagatgtcatgaggcgagctgggattaaggtcaccgttgcaggcctggctggaaaagacccagtacagtgtagccgtgatgtggtcatttgtcctgatgccagccttgaagatgcaaaaaaagagggaccatatgatgtggtggttctaccaggaggtaatctgggcgcacagaatttatctgagtctgctgctgtgaaggagatactgaaggagcaggaaaaccggaagggcctgatagccgccatctgtgcaggtcctactgctctgttggctcatgaaataggttttggaagtaaagttacaacacaccctcttgctaaagacaaaatgatgaatggaggtcattacacctactctgagaatcgtgtggaaaaagacggcctgattcttacaagccgggggcctgggaccagcttcgagtttgcgcttgcaattgttgaagccctgaatggcaaggaggtggcggctcaagtgaaggctccacttgttcttaaagactagagcagcgaactgcgacgatcacttagagaaacaggccgttaggaatccattctcactgtgttcgctctaaacaaaacagtggtaggttaatgtgttcagaagtcgctgtccttactacttttgcggaagtatggaagtcacaactacacagagatttctcagcctacaaattgtgtctatacatttctaagccttgtttgcagaataaacagggcatttagcaaactactgattgtttcttgttttgtctctcatttcttttgtgaaattaaattccgtatcaccttcatttgcagctcttaactgtccatatggcactgaaataaaagaacagtgaccacattttacacagcaaggaggaaaggcatacaaacagaatttaagaggcttgtgattttctctgcttattagctgtgtgtttttaatgtgctattaaaaaataccaatgagg

SEQ ID NO: 3 - protein, 139 aa, Mus musculus
KVTVAGLAGKDPVQCSRDVMICPDTSLEDAKTQGPYDVVVLPGGNLGAQNLSESPMVKEILKEQESRKGLIAAICAGPTALLAHEVGFGCKVTTHPLAKDKMMNGSHYSYSESRVEKDGLILTSRGPGTSFEFALAIVE

SEQ ID NO: 4 - protein, 189 aa, Mus musculus
MASKRALVILAKGAEEMETVIPVDVMRRAGIKVTVAGLAGKDPVQCSRDVMICPDTSLEDAKTQGPYDVVVLPGGNLGAQNLSESPMVKEILKEQESRKGLIAAICAGPTALLAHEVGFGCKVTTHPLAKDKMMNGSHYSYSESRVEKDGLILTSRGPGTSFEFALAIVEALVGKDMANQVKAPLVLKD

SEQ ID NO: 5 - RNA, 920 nt, Mus musculus
  source 1..920: mol_type = mRNA
tgacgcaggccgaggcggaagcgaggggttgtgcgcagcctcagtgcgggtggcgcgcatgcgtgctgggtacacgtcgggtgcgaggttcctgcggactagcggtggcttcgcgtgggtggaggaggcgcggctgcagtctttaagaaatagaaatggcttccaaaagagctctggtcatcctggccaaaggagcagaggagatggagacagtgattcctgtggatgtcatgcggcgagccgggatcaaagtcactgttgcaggcttggctgggaaggaccccgtgcagtgtagccgtgatgtaatgatttgtccagataccagtctggaagatgcaaaaacgcagggaccatacgatgtggtggttcttccaggaggaaatctgggtgcacagaatttatctgagtcgcctatggtgaaggagatcctcaaggagcaggagagcaggaagggcctcatagctgccatctgtgcaggtcctacggctctgttggctcacgaagtaggttttggatgcaaggtcacaacacacccactggctaaggacaaaatgatgaatggcagtcactacagctactcagagagccgcgtggagaaggacggcctgatcctcaccagccgcgggccggggaccagctttgagtttgcactagccattgtggaggcactcgtggggaaagacatggccaaccaagtgaaggcaccgcttgttctcaaagactagagcccaagccctgggccccacgcttgagcaggcattggaagcccactggtgtgtccagagcccagggaacctcagcagtagtatgtgaagcagccgccacacggggctctcatcccgggtctgtatgtttctgaaccttgctagtagaataaacagtttaccaagctcctgccagctaaaaaaaaaaaaaaaaaa

(19) United States
(12) Patent Application Publication
(10) Pub. No.: US 2025/0339501 A1
(43) Pub. Date: Nov. 6, 2025

(54) COMPOSITIONS AND METHODS FOR TREATING METABOLIC DISEASE

(71) Applicant: The Regents of the University of California, Oakland, CA (US)

(72) Inventor: Zhenqi Zhou, Los Angeles, CA (US)

(21) Appl. No.: 18/698,812

(22) PCT Filed: Oct. 4, 2022
(86) PCT No.: PCT/US22/45615

§ 371 Date: Apr. 5, 2024

Related U.S. Application Data
(60) Provisional application No. 63/252,443, filed on Oct. 5, 2021.

(51) Int. Cl. A61K 38/50; A61K 31/155; A61K 38/26; A61K 38/28; A61K 45/06; A61P 1/16; C12Q 1/6883; G01N 33/68

(52) U.S. Cl. CPC A61K 38/50; A61K 31/155; A61K 38/26; A61K 38/28; A61K 45/06; A61P 1/16; C12Q 1/6883; G01N 33/6893; C12Q 2600/158; C12Y 305/01; G01N 2333/98; G01N 2800/10; G01N 2800/50; G01N 2800/56

(57) ABSTRACT

The present disclosure relates to compositions and methods for treating obesity-related diseases. In some aspects, methods are provided for increasing the expression and/or activity of Dj 1 protein in a subject in need thereof.

### CROSS-REFERENCE TO RELATED APPLICATION

This application claims the benefit of U.S. Provisional Patent Application No. 63/252,443, filed on Oct. 5, 2021, which is hereby incorporated by reference in its entirety.

### STATEMENT OF RIGHTS TO INVENTIONS MADE UNDER FEDERALLY SPONSORED RESEARCH

This invention was made with government support under Grant No. DK063491 awarded by the National Institutes of Health. The government has certain rights in the invention.

### SEQUENCE LISTING

The instant application contains a Sequence Listing which has been submitted via Patent Center and is hereby incorporated by reference in its entirety. Said .xml copy, created on Apr. 30, 2024 is named UCH-28801, and is 7,893 bytes in size.

### BACKGROUND

Metabolic dysfunction, manifested clinically as metabolic syndrome, has become a global epidemic that dramatically increases the risk of non-alcoholic fatty liver disease (NAFLD), type 2 diabetes, and cardiovascular disease (CVD). Elevated hepatic glucose production and lipoprotein secretion contribute to the pathogenesis of hyperglycemia and hyperlipidemia in insulin resistance, and obesity is associated with excess fat accumulation in the liver, a defining feature of NAFLD that affects one-third of adults and an increasing number of children in developed countries (1). NAFLD refers to a group of related disorders increasing in severity from hepatic steatosis, nonalcoholic steatohepatitis (NASH), cirrhosis, and ultimately hepatocarcinomas (1,2). Although combination and single drug therapies are under evaluation, no pharmacological treatment is approved for NAFLD (2). Hence, understanding the molecular mechanisms underlying NAFLD and hormonal cues that mediate the crosstalk among different tissues, particularly between skeletal muscle and the liver are urgent for the development of effective therapies.

The foregoing observations provide evidence of the continuing need for compositions and formulations useful in treating metabolic dysfunction diseases, such as type 2 diabetes, cardiovascular disease, and NAFLD.

### SUMMARY OF THE INVENTION

The invention features compositions and methods that are useful for diagnosing muscle injury and treating metabolic diseases and disorders. The invention is based, at least in part, on the discovery that skeletal muscle releases Dj1 protein in extracellular vesicles and that Dj1 enhances fatty acid oxidation, decreases lipid content, improves mitochondrial function, and suppresses the ASK1-JNK-PPARα signaling in the liver and improves glucose homeostasis in non-alcoholic steatohepatitis in mice.

Accordingly, one aspect of the invention provides a method of treating a metabolic disease or disorder or symptom thereof, comprising administering to a patient in need thereof a therapeutically effective amount of a Dj1 polypeptide or a fragment thereof or polynucleotide encoding a Dj1 polypeptide or a fragment thereof. The metabolic disorder can be selected from non-alcoholic fatty liver disease (NAFLD), steatohepatitis, type II diabetes, hyperglycemia, hyperlipidemia, dyslipidemia, obesity, hyperinsulinemia, insulin resistance, hypercholesterolemia, non-familial hypercholesterolemia, familial hypercholesterolemia, heterozygous familial hypercholesterolemia, homozygous familial hypercholesterolemia, mixed dyslipidemia, atherosclerosis, early onset coronary heart disease, dyslipidemia, hypertriglyceridemia, hyperfattyacidemia, and cirrhosis. The fragment of the Dj1 polypeptide can comprise residues 32-173. The Dj1 polypeptide or polypeptide or fragment thereof can be purified and/or isolated from recombinant sources.

### BRIEF DESCRIPTION OF THE DRA WINGS

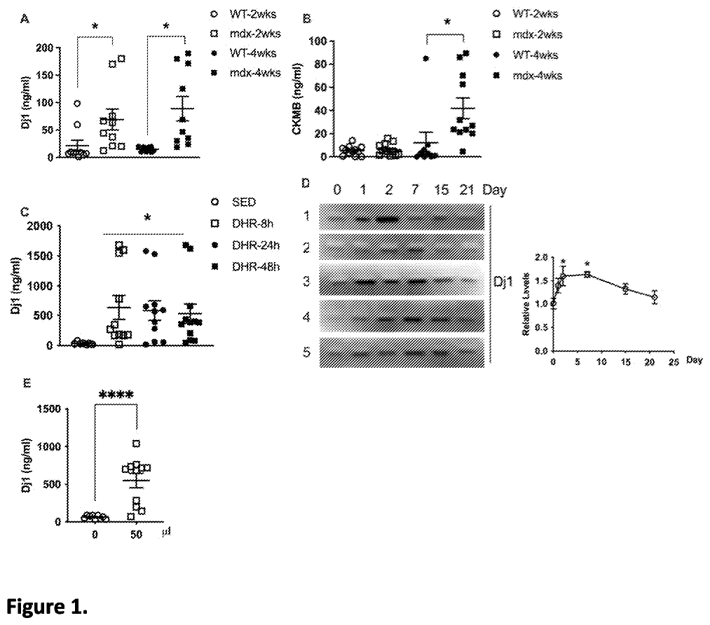

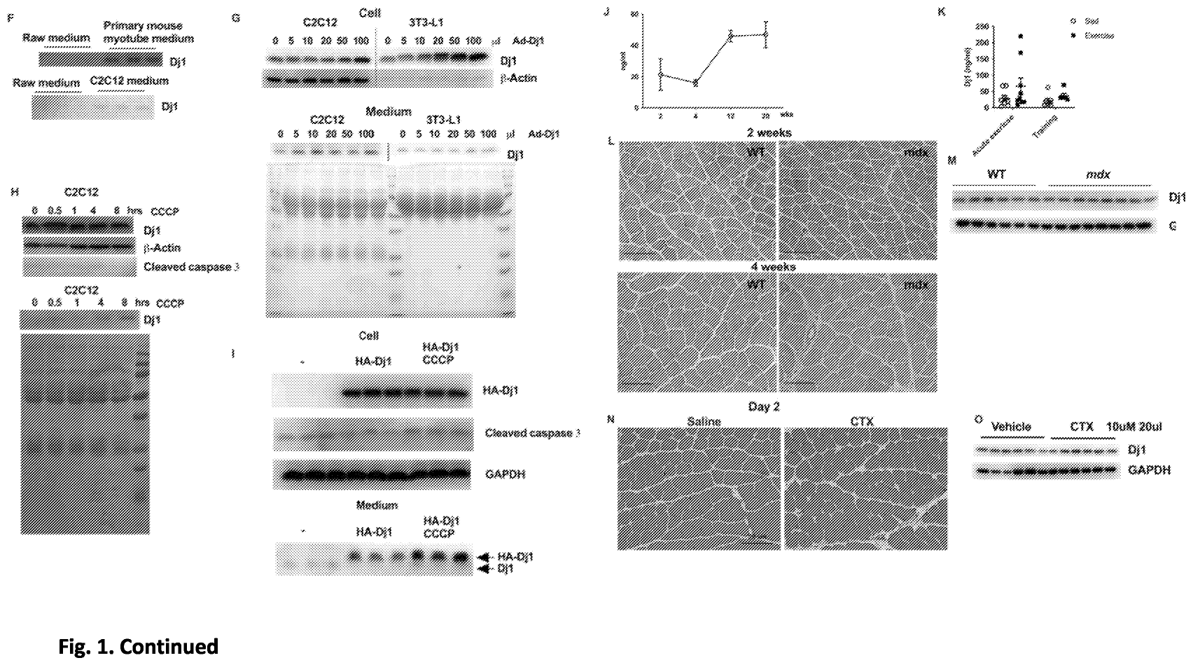

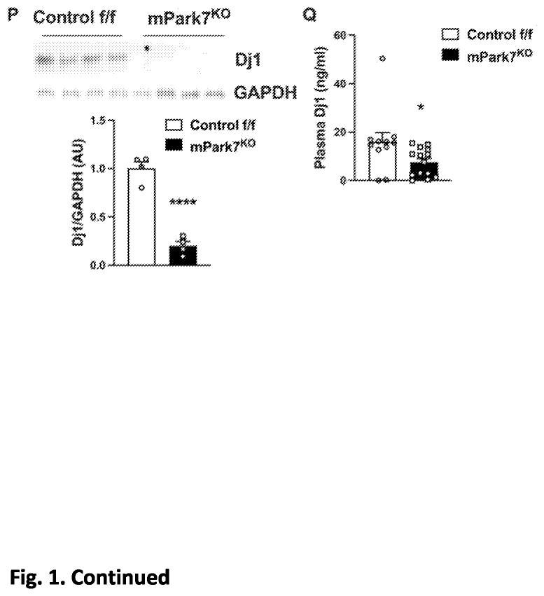

FIGS. 1A-1Q show that skeletal muscle injury elevates the circulating levels of Dj1. FIGS. 1A and 1B show circulating levels of Dj1 and CKMB, respectively, of 2 and 4 weeks-old mdx mice (n=8-10). FIG. 1C shows plasma Dj1 levels of the mice after down-hill running (DHR) at the indicated time vs. the sedentary mice (SED) (n=7-12). FIG. 1D shows that the circulating levels of Dj1 from CTX-injected wild-type mice (WT) were analyzed via immunoblotting. The bar graph on the right is the protein densitometry analysis of Dj1 in the plasma (n=5). FIG. 1E shows plasma Dj1 levels of WT mice intramuscularly (IM) injected with CTX (Day 2 post-injection). FIG. 1F shows an immunoblot analysis of Dj1 protein in the raw culture medium (medium only) and the culture medium of primary mouse myotubes and C2C12 myotubes (n=3). FIG. 1G comprises immunoblot analyses of Dj1 protein in C2C12 myocytes and 3T3-L1 preadipocytes that were transduced with adenovirus encoding a Dj1 protein at the indicated dosage and in the cell media of these cultures. FIG. 1H is an immunoblot analysis of Dj1 protein in C2C12 myocytes treated with carbonyl cyanide m-chlorophenyl hydrazine (CCCP) at the indicated time and an immunoblot showing the Dj1 protein level in the culture medium. FIG. 1I is an immunoblot analysis of Dj1 protein level in C2C12 myocytes and in the culture media with and without CCCP treatment. FIG. 1J is a graph showing the circulating Dj1 protein level in male wild-type mice at the indicated age (n=5-10). FIG. 1K is a plot showing the plasma Dj1 levels of WT mice that underwent acute exercise or exercise training and WT mice that were kept sedentary (n=5-10). FIG. 1L includes images of H & E staining of gastrocnemius muscle sections from 2-week-old and 4-weeks old WT mice and mdx mice. FIG. 1M is an immunoblot analysis of Dj1 protein level in skeletal muscle of 2-week-old WT mice and mdx mice (n=6-8). FIG. 1N shows images of H & E staining of gastrocnemius muscle from WT mice injected with saline or CTX (n=6). FIG. 1O is an immunoblot analysis of the Dj1 protein levels of gastrocnemius muscle from WT mice injected saline or CTX (n=6). FIGS. 1P-1Q show skeletal muscle is the primary source of circulating Dj1. FIG. 1P is an immunoblot analysis of the Dj1 protein levels of gastrocnemius muscle from Control f/f vs. mPark7^{KO} mice (n=4). FIG. 1Q shows the plasma Dj1 levels in Control f/f vs. mPark7^{KO} mice (n=12-14). Values are mean±SEM, *=p<0.05.

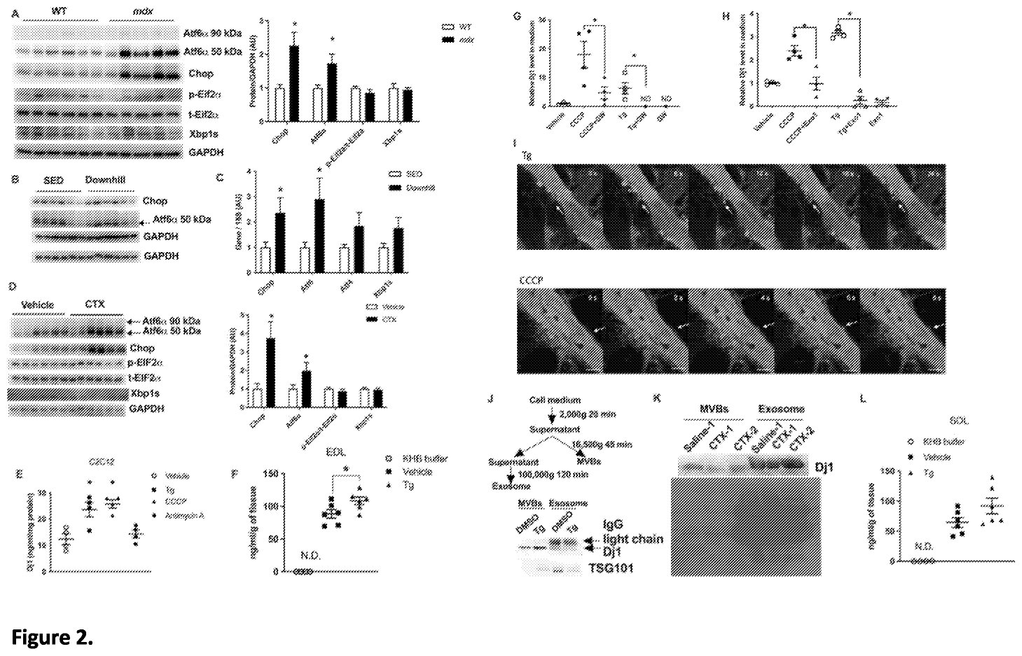

FIGS. 2A-2L show that endoplasmic reticulum (ER) stress stimulates muscle cells release of Dj1. FIG. 2A shows an immunoblotting analysis of ER stress markers in quadriceps muscle of WT and mdx mice (n=5-6). Bar graphs on the right shows the densitometry of ER stress markers that were normalized to GAPDH. FIGS. 2B and 2C are the protein levels and the gene expression of ER stress markers in the quadriceps muscle of the SED and downhill-running (DHR) C57BL/6J mice (n=6). FIG. 2D is an immunoblot analysis of ER stress marker expression in C57BL/6J mice that were administered with vehicle or 10 μM CTX (n=6). Bar graphs on the right shows the densitometry of ER stress markers that were normalized to GAPDH. FIG. 2E is a plot showing the protein levels of Dj1 in the cell culture medium from C2C12 myotubes treated with vehicle, thapsigargin (Tg), CCCP, or antimycin A (n=4). FIG. 2F is a plot showing the protein levels of Dj1 in the Krebs-Henseleit buffer (KHB) only and the KHB medium of EDL muscle treated with vehicle or Tg (n=6). FIG. 2G is a plot showing the relative Dj1 levels in the medium of C2C12 myotubes treated with vehicle, CCCP, CCCP+GW4869, Tg, Tg+GW4869, or GW4869 (n=4). FIG. 2H is a plot showing the relative Dj1 levels in the medium of C2C12 myotubes treated with vehicle, CCCP, CCCP+Exo1, Tg, Tg+Exo1, or Exo1 (n=4-6). FIG. 2I is time-lapse images of Dj1-GFP overexpressed C2C12 myocytes treated with Tg or CCCP. FIG. 2J is a schematic of an experimental design to separate multivesicular bodies (MVBs) and exosomes from the cell culture medium. The immunoblot analysis shows that Dj1 protein is enriched in the multivesicular bodies (MVBs) of the cell medium from C2C12 myotubes treated with Tg. FIG. 2K is an immunoblot analysis showing that Dj1 was detected in both MVBs and exosomes of WT mice plasma. FIG. 2L is a plot showing Dj1 protein level in the Krebs-Henseleit buffer (KHB) only and the KHB medium of soleus (SOL) muscle treated with vehicle or Tg (n=4-6). Values are mean±SEM, *=p<0.05.

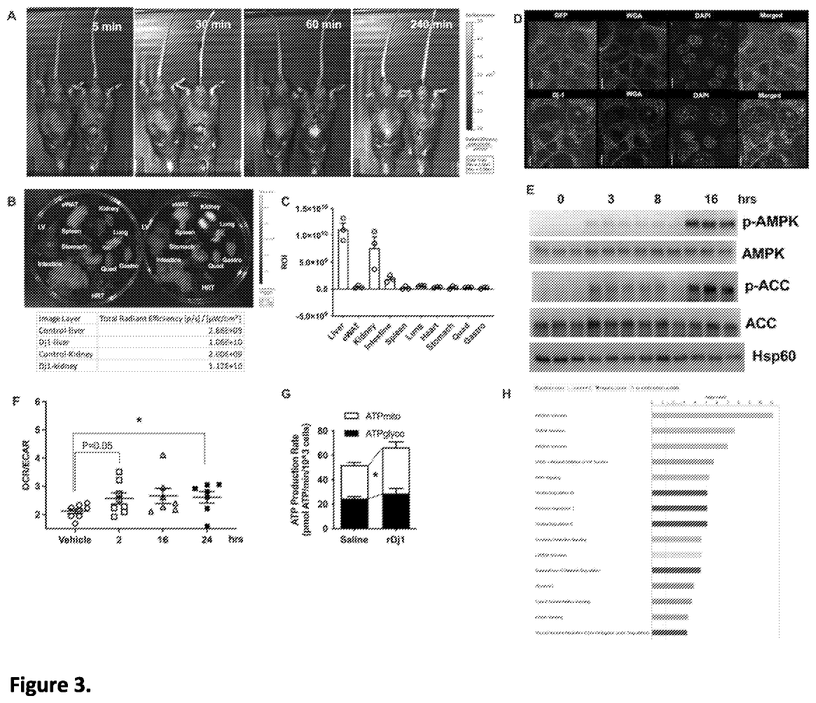

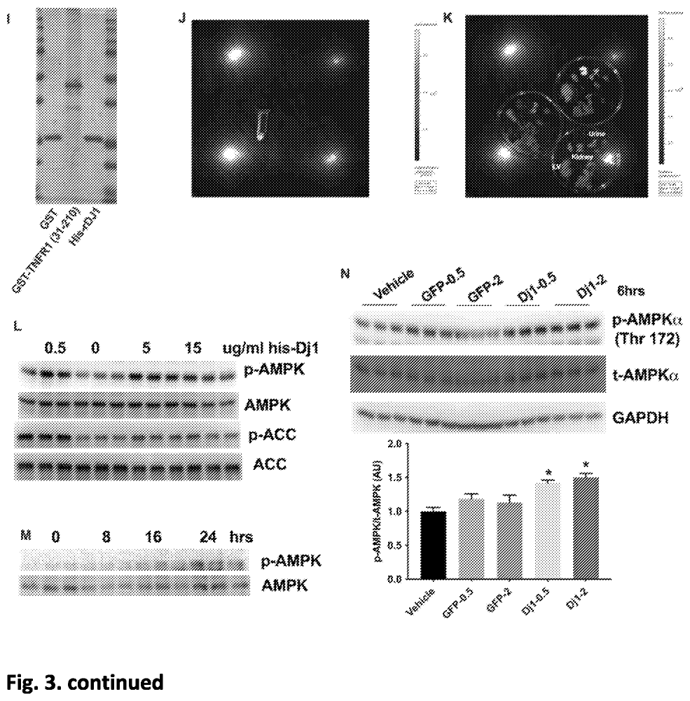

FIGS. 3A-3N show that Dj1 targets the liver and kidneys of the mice. FIG. 3A shows intravital imaging of WT mice that were administered with IRDye 800CW-labeled Dj1 via tail vein (IV) injection. FIG. 3B and FIG. 3C show images and region of interest (ROI) quantification, respectively, of IRDye 800CW-labeled Dj1 signals in organs. FIG. 3D shows fluorescent imaging of AML12 mouse liver cells treated with GFP or Alexa Fluor 488 labeled Dj1 protein (without wash). Plasma membranes were stained with Wheat Germ Agglutinin conjugated to Alexa Fluor 594. Nuclei were stained with DAPI. FIG. 3E is a immunoblot analysis showing that recombinant human Dj1 protein activates the phosphorylation of AMPK and ACC in HepG2 cells (n=3). FIG. 3F is a plot showing recombinant human Dj1 protein enhances OCR/ECAR ratio. FIG. 3G is a graph showing recombinant human Dj1 protein enhances ATP production from mitochondria (n=6-8). FIG. 3H shows an Ingenuity Pathway Analysis (IPA) of RNA sequencing data of HepG2 cells treated with saline or tDj1 (n=4). FIG. 3I shows Coomassie blue staining of purified His-rDj1 protein (the second lane from right). FIG. 3J shows imaging of IRDye 800CW-labeled Dj1 protein in a tube. FIG. 3K shows imaging of dissected organs from mice injected with saline (left dish) and IRDye 800CW-labeled Dj1 (right two dishes). FIG. 3L is an immunoblot analysis of phosphorylated and total AMPK and ACC in HepG2 cells treated with His-Dj1 protein at the indicated dosage (n=3). FIG. 3M is an immunoblot analysis of phosphorylated and total AMPK in primary mouse hepatocytes treated with His-Dj1 protein at the indicated time (n=3). FIG. 3N is an immunoblot analysis of phosphorylated and total AMPK in HepG2 cells treated with GFP or His-Dj1 protein at the indicated dosage and time (n=3). Values are mean±SEM, *=p<0.05.

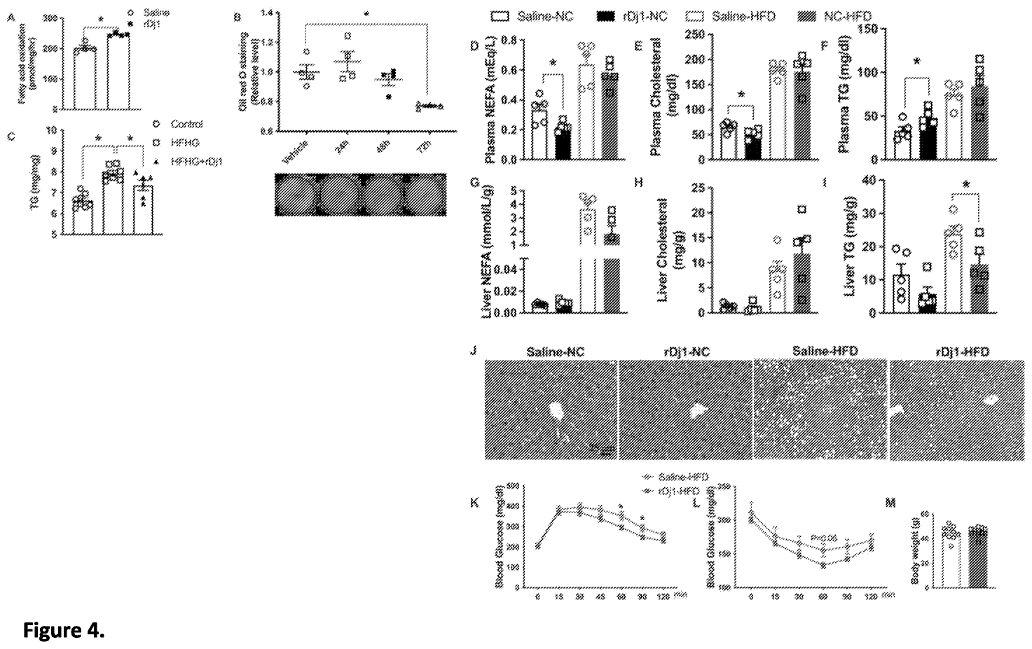

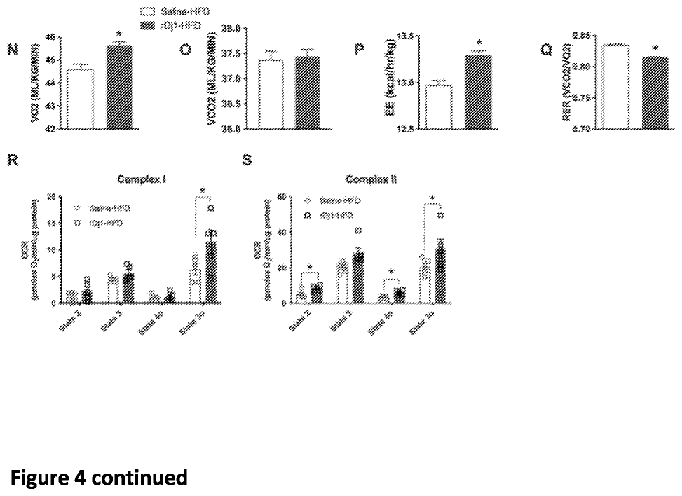

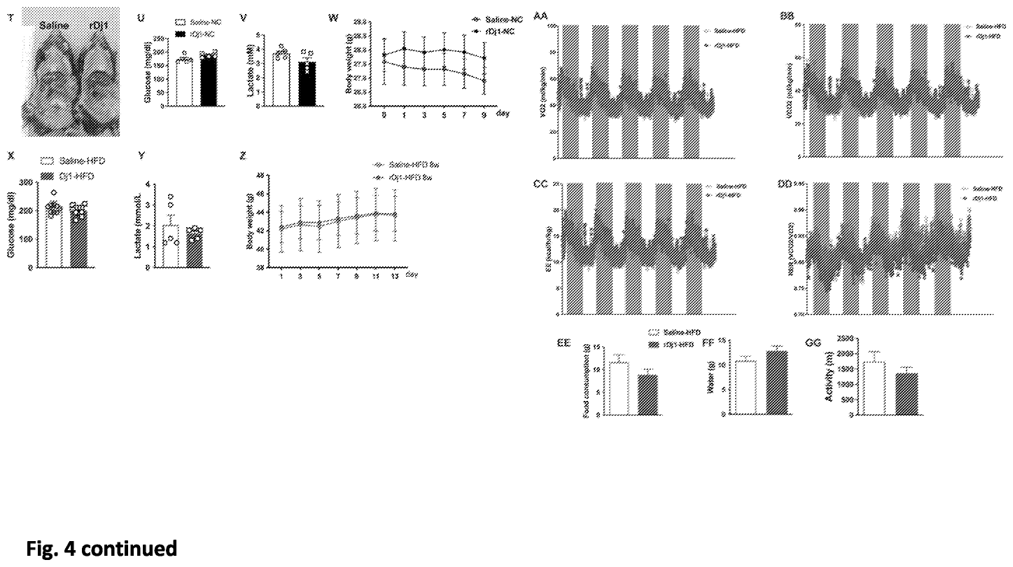

FIGS. 4A-4GG show that Dj1 administration improves lipid metabolism and glucose homeostasis. FIG. 4A is a graph quantifying fatty acid oxidation in HepG2 cells treated with saline or recombinant human Dj1 protein (rDj1) (n=4). FIG. 4B is a graph quantifying Oil red O staining of HepG2 cells treated with rDj1 at the indicated time (n=4). FIG. 4C shows the triacylglycerol (TG) level in HepG2 cells cultured in low glucose medium (Control), high-fat high-glucose (HFHG), and high-fat high-glucose plus rDj1 (HFHD+rDj1) (n=6-8). The data presented in FIGS. 4D-4I are from experiments with C57BL/6J mice that were administered mouse recombinant Dj1 (rDj1) protein (0.15 mg/kg/day via IV for 5 days; n=5). FIG. 4D is a graph showing plasma NEFA levels. FIG. 4E is a graph showing plasma cholesterol levels. FIG. 4F is a graph showing plasma TG levels. FIG. 4G is a graph showing liver NEFA. FIG. 4H is a graph showing liver cholesterol levels. FIG. 4I is a graph showing liver TG levels. FIG. 4J includes images of H & E staining of liver sections from NC or HFD-fed mice injected with saline or rDj1. The data presented in FIGS. 4K-4M are from experiments with HFD-fed C57BL/6J mice that were administered with rDj1 (0.15 mg/kg/day; IV) for 5 days (n=10-11). FIG. 4K is a graph quantifying the results of a glucose tolerance test of the HFD-fed C57BL/6J mice after treatment. FIG. 4L is a graph quantifying the results of an insulin tolerance test of the HFD-fed C57BL/6J mice after treatment. FIG. 4M is a graph showing the body weight of the HFD-fed C57BL/6J mice after treatment. The data presented in FIGS. 4N-4S are from experiments on HFD-fed mice that were administered with either saline or rDj1 (n=4-5). FIG. 4N is a graph showing oxygen consumption (VO₂) in the HFD-fed mice. FIG. 4O is a graph showing CO₂ production (VCO₂) in the HFD-fed mice. FIG. 4P is a graph showing energy expenditure (EE) in the HFD-fed mice. FIG. 4Q is a respiratory exchange ratio (RER) in the HFD-fed mice. FIG. 4R and FIG. 4S are graphs showing mitochondrial complex I and complex II activity, respectively, of isolated mitochondria from the liver of the HFD-fed mice treated with saline vs. rDj1 (n=5). FIG. 4T is an image of mice that were administered with saline or rDj1. FIG. 4U is a graph showing fasting blood glucose levels of NC-fed mice that were administered with saline or rDj1 via IV injection (n=5). FIG. 4V is a graph showing lactate concentration in lactate of NC-fed mice that were administered with saline or rDj1 via IV injection (n=5). FIG. 4W is a graph of body weight of NC-fed mice that were administered with saline or rDj1 (n=5). FIG. 4X is a graph of fasting blood glucose levels of HFD-fed mice that were administered with saline or rDj1 (n=5). FIG. 4Y is a graph showing lactate concentration in lactate of HFD-fed mice that were administered with saline or rDj1 (n=5). FIG. 4Z is a graph showing body weight of HFD-fed mice administered with saline or rDj1 (n=5). The data presented in FIGS. 4AA-4GG were obtained from experiments in HFD-fed mice that were administered with saline or rDj1 (n=4-5). FIG. 4AA is a graph showing oxygen consumption (VO₂) in the HFD-fed mice. FIG. 4BB is a graph showing CO₂ production (VCO₂) in the HFD-fed mice. FIG. 4CC is a graph showing energy expenditure (EE) in the HFD-fed mice. FIG. 4DD is a graph showing respiratory exchange ratio (RER) of the HFD-fed mice. FIG. 4EE is a graph showing food consumption in the HFD-fed mice. FIG. 4FF is a graph showing water consumption in the HFD-fed mice. FIG. 4GG is a graph showing total activity of the HFD-fed mice. Values are mean±SEM, *=p<0.05.

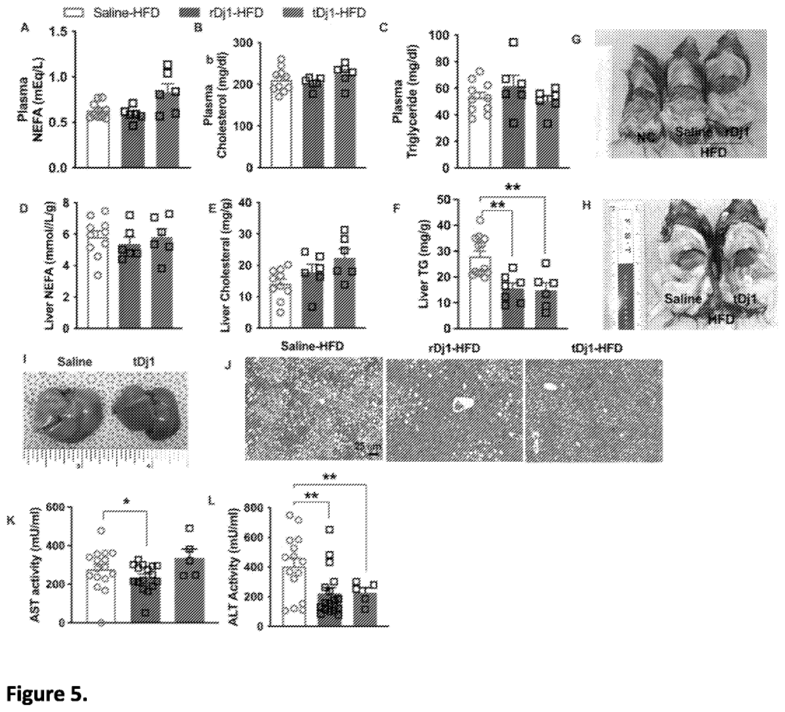

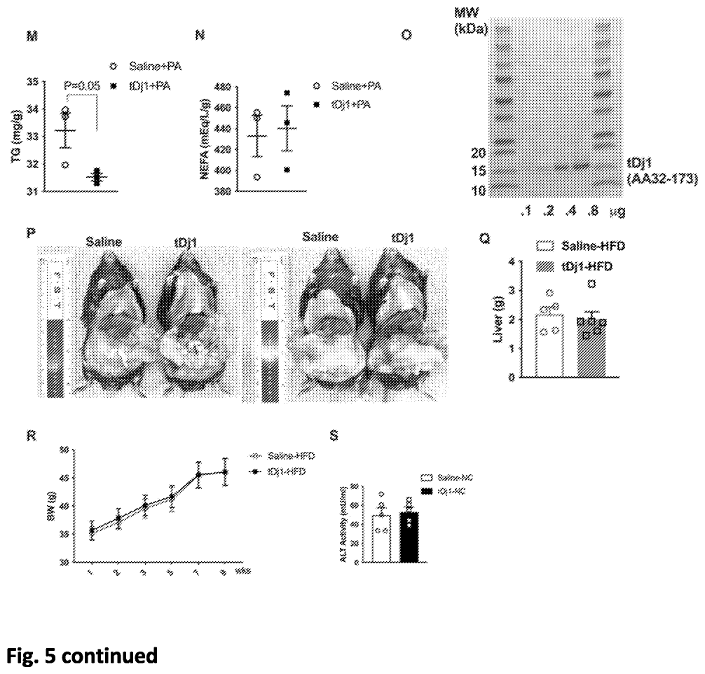

FIGS. 5A-5S show that Dj1 administration reduces hepatic steatosis. FIGS. 5A-5F are graphs comparing biomarker levels in HFD-fed mice administered with saline, rDj1, or tDj1 (n=6-10). FIG. 5A is a graph showing plasma NEFA levels. FIG. 5B is a graph showing plasma cholesterol levels. FIG. 5C is a graph showing plasma triglyceride levels. FIG. 5D is a graph showing liver NEFA levels. FIG. 5E is a graph showing liver cholesterol levels. FIG. 5F is a graph showing liver triglyceride levels. FIG. 5G is an image of HFD-fed mice that were administered with saline or rDj1. FIG. 5H is an image of HFD-fed mice that were administered with saline or tDj1. FIG. 5I is an image of the liver from HFD-fed mice that were administered with saline or tDj1. FIG. 5J includes images of H & E staining of liver sections from HFD-fed mice that were administered with saline, rDj1, or tDj1. FIG. 5K is a graph of aspartate aminotransferase (AST) activity in the liver of HFD-fed mice (n=5-17) that were administered with saline, rDj1, or tDj1 via injection. FIG. 5L is a graph alanine transaminase (ALT) activity in the liver of HFD-fed mice (n=5-17) that were administered with saline, rDj1, or tDj1 via tail-vein injection. FIG. 5M is a graph showing TG levels in AML12 cells treated with saline+PA or tDj1+PA (n=3). FIG. 5N is a graph of NEFA content in AML12 cells treated with saline+PA or tDj1+PA (n=3). FIG. 5O is a Coomassie blue staining of purified His-tDj1 protein. FIG. 5P are the images of the liver from HFD-fed mice administered with saline or tDj1. FIG. 5Q is a graph showing the average liver weight of HFD-fed mice treated with saline or tDj1. FIG. 5R is a graph of body weight of HFD-fed mice during the rDj1 injection (n=5-6). FIG. 5S is a graph showing ALT activity in the liver of NC-fed mice treated with saline or rDj1 (n=5). Values shown in FIGS. 5A-5F, 5K-5N, and 5Q-5S are mean±SEM, *=p<0.05, **=p<0.01.

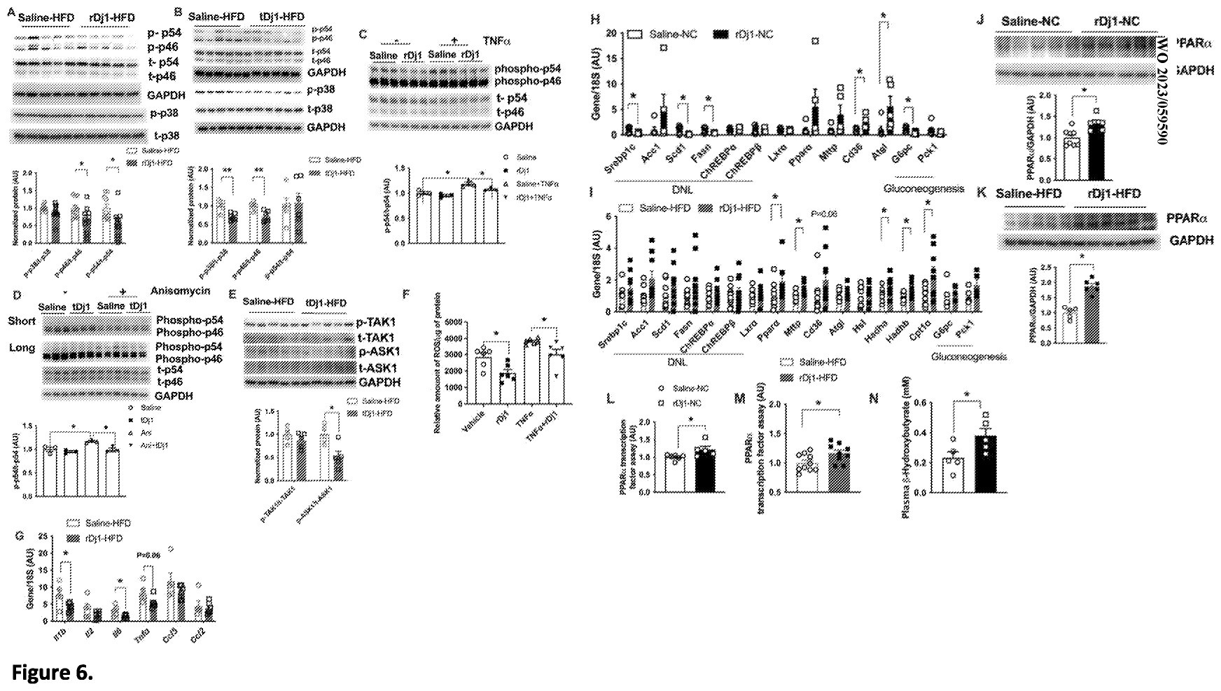

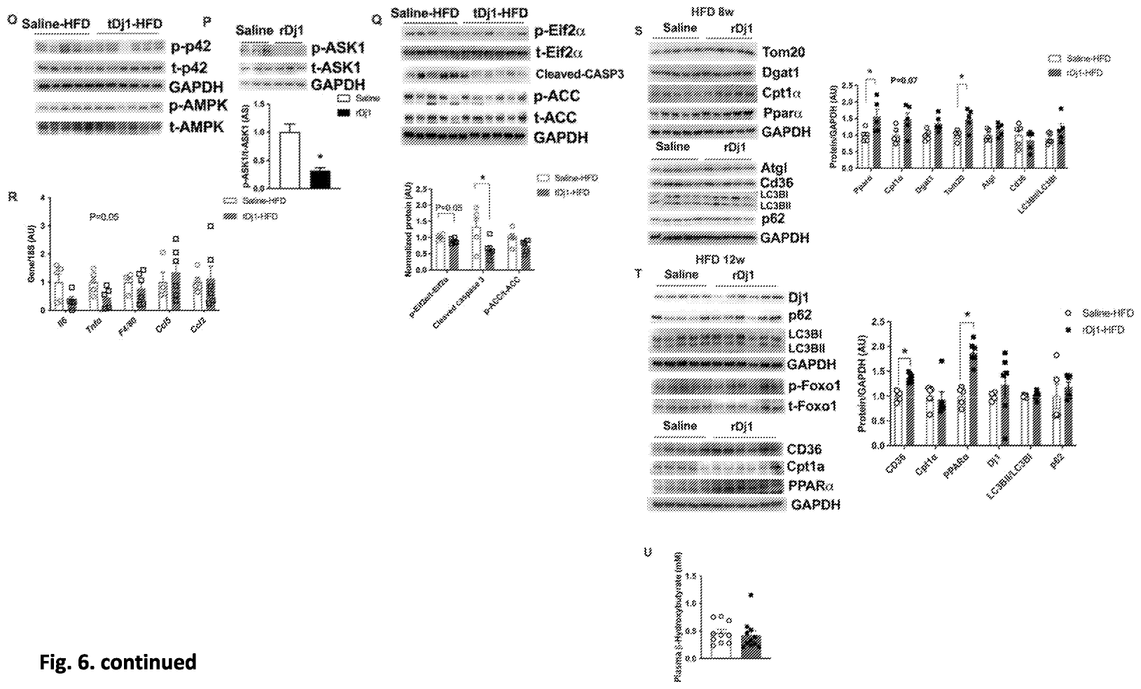

FIGS. 6A-6U show that both rDj1 and truncated Dj1 (tDj1) impact hepatic metabolism through the ASK1-JNK-PPARα pathway. FIG. 6A and FIG. 6B are immunoblot analyses of the protein levels in the JNK (p-54 and p-46) and p38 MAPK pathways, respectively, in HFD-fed mice (n=5) that were administered with saline, rDj1, or tDj1. FIG. 6C shows that rDj1 inhibits TNFα-induced JNK phosphorylation in HepG2 cells (n=3). FIG. 6D shows that tDj1 inhibits Anisomycin-induced JNK phosphorylation in AML12 cells (n=3). FIG. 6E shows that tDj1 inhibits the phosphorylation of ASK1, but not TAK1 in the liver of HFD-fed WT mice (n=5). The bar graph at the bottom is the protein densitometry analysis. FIG. 6F is a graph showing the relative amount of reactive oxygen species (ROS) in HepG2 cells treated with vehicle, rDj1, TNFα, or TNFα+rDj1 (n=6). FIG. 6G is a graph showing proinflammatory cytokine gene expression in the liver of HFD-fed mice that were administered with saline or rDj1 (n=5-6). FIG. 6H and FIG. 6I are graphs showing gene expression in the liver of NC-fed mice (n=5-7) and HFD-fed mice (n=10), respectively, that were treated with saline or rDj1. FIG. 6J and FIG. 6K are immunoblot analyses of PPARα expression in the liver of NC-fed mice (n=5) and HFD-fed mice (n=5-6), respectively, that were treated with saline or rDj1. FIG. 6L and FIG. 6M are graphs showing PPARα transcriptional activity in the liver of NC-fed mice (n=5-6) and HFD-fed mice (n=9-10), respectively, that were treated with saline or rDj1 (n=9-10). FIG. 6N is a graph showing the plasma beta-hydroxybutyrate level of NC-fed mice (n=5). FIG. 6O is an immunoblot analysis of p-p42/t-p42 and p-AMPK/t-AMPK in the liver of HFD-fed mice treated with saline or tDj1 (n=5-6). FIG. 6P shows phosphorylated and total ASK1 protein levels in HepG2 cells treated with saline or rDj1. FIG. 6Q is an immunoblot analysis of Eif2α, cleaved caspase 3, and ACC in the liver of HFD-fed mice treated with saline or tDj1 (n=5-6). FIG. 6S is an immunoblot analysis of autophagy and lipid metabolism proteins in the liver of 8 wks and 12 wks HFD-fed mice treated with saline or rDj1 (n=5-6). FIG. 6T is an immunoblot analysis of the autophagy and lipid metabolism proteins in the liver of 12 wks HFD-fed mice treated with saline or rDj1 (n=5-6). FIG. 6U is a graph showing the plasma beta-hydroxybutyrate level of the HFD-fed mice treated with saline or rDj1 (n=10-11). Values are mean±SEM, *=p<0.05.

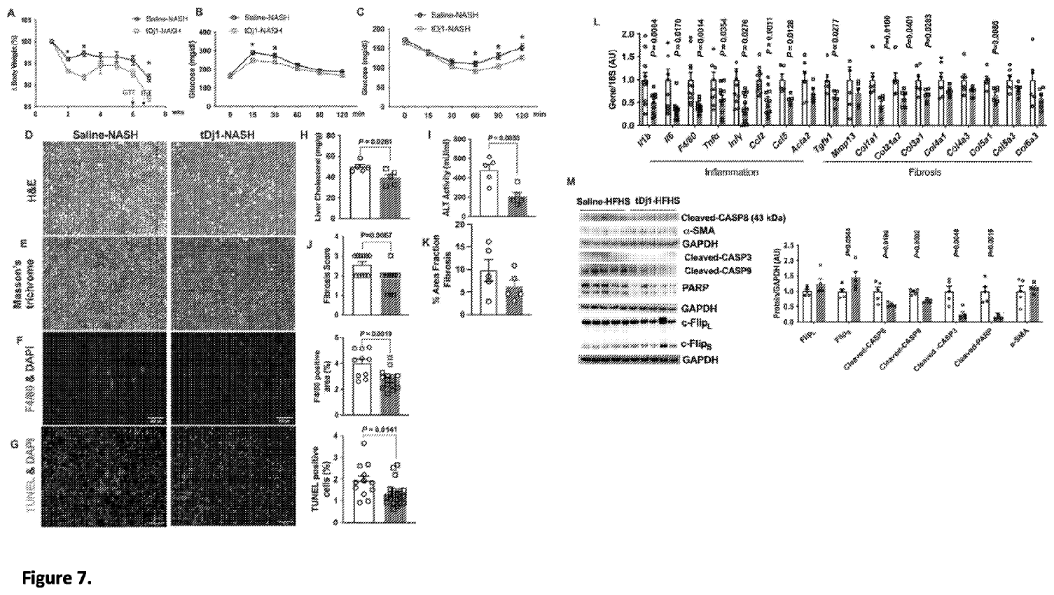

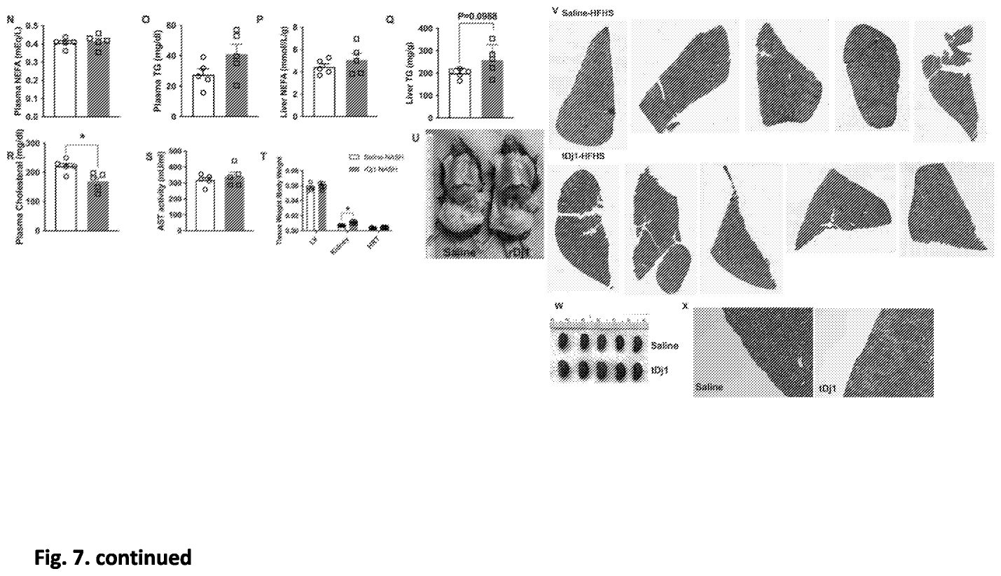

FIGS. 7A-7X show that Dj1 improves glucose and insulin sensitivity and suppresses inflammation and apoptosis in the liver of diet-induced mouse NASH model. The data presented in FIGS. 7A-7M were obtained from NASH diet-fed mice (n=5-10) treated with saline or tDj1. FIG. 7A is a graph showing the change of body weight in these mice. FIG. 7B is a graph quantifying glucose tolerance in these mice. FIG. 7C is a graph quantifying insulin tolerance in these mice. FIG. 7D includes the images of H & E stained liver tissues obtained from these mice. FIG. 7E includes the images of liver tissues obtained from these mice and stained with Masson's trichrome. FIG. 7F includes images of liver tissues obtained from these mice and labeled with fluorescently labeled anti-F4/80 antibodies and DAPI stained. FIG. 7G includes images of liver tissues obtained from these mice and subjected to TUNEL and DAPI staining. FIG. 7H is a graph showing liver cholesterol levels in the NASH diet-fed mice treated with saline or tDj1 (n=5). FIG. 7I is a graph showing ALT activity in the liver of these mice (n=5). FIG. 7J is a graph showing the liver fibrosis score (n=11). FIG. 7K is a graph showing the percentage area fraction of liver fibrosis for these mice (n=5). FIG. 7L is a graph showing a qPCR analysis of inflammation and fibrosis gene expression in the liver of NASH diet-fed mice treated with saline or tDj1 (n=5-11). FIG. 7M is an immunoblotting analysis of apoptosis markers. FIG. 7N, FIG. 7O, FIG. 7P, FIG. 7Q, FIG. 7R, FIG. 7S, and FIG. 7T are graphs showing plasma NEFA, plasma TG, liver NEFA, liver TG, plasma cholesterol, AST activity, and tissue weight/body weight ratio of NASH diet-fed mice treated with saline or rDj1 (n=5). Values are mean±SEM, *=p<0.05.

### DETAILED DESCRIPTION

The invention features compositions and methods that are useful for diagnosing muscle injury and treating metabolic diseases and disorder, such as non-alcoholic fatty liver disease (NAFLD), steatohepatitis, type II diabetes, hyperglycemia, hyperlipidemia, dyslipidemia, obesity, hyperinsulinemia, insulin resistance, hypercholesterolemia, non-familial hypercholesterolemia, familial hypercholesterolemia, heterozygous familial hypercholesterolemia, homozygous familial hypercholesterolemia, mixed dyslipidemia, atherosclerosis, early onset coronary heart disease, dyslipidemia, hypertriglyceridemia, hyperfattyacidemia, and cirrhosis. The invention is based, at least in part, on the discovery that skeletal muscle releases of Dj1 in extracellular vesicles and that Dj1 enhances fatty acid oxidation, decreases lipid content, improves mitochondrial function, and suppresses the ASK1-JNK signaling in the liver and improves glucose homeostasis in non-alcoholic steatohepatitis in mice.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

By “agent” is meant a peptide, nucleic acid molecule, or small compound.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels. “

In this disclosure, “comprises,” “comprising,” “containing,” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence, or amount of the analyte to be detected.

By “detectable label” is meant a composition that when linked to a molecule of interest renders the latter detectable via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxygenin, or haptens.

“Dj1 polynucleotide” refers to a nucleic acid molecule that encodes a Dj1 polypeptide. For example, SEQ ID NOs: 2 and 5 are nucleic acid sequences of Dj1 polynucleotides, and SEQ ID NOs: 1, 3, and 4 are amino acid sequences of Dj1 polypeptides. Thus, the phrase “Dj1 polypeptide or polynucleotide encoding a Dj1 polypeptide or fragment thereof” refers to a Dj1 polypeptide or a Dj1 polynucleotide.

By “disease” is meant any condition or disorder that damages or interferes with the normal function of a cell, tissue, or organ. Examples of diseases include non-alcoholic fatty liver disease (NAFLD), steatohepatitis, type II diabetes, hyperglycemia, hyperlipidemia, dyslipidemia, obesity, hyperinsulinemia, insulin resistance, hypercholesterolemia, non-familial hypercholesterolemia, familial hypercholesterolemia, heterozygous familial hypercholesterolemia, homozygous familial hypercholesterolemia, mixed dyslipidemia, atherosclerosis, early onset coronary heart disease, dyslipidemia, hypertriglyceridemia, hyperfattyacidemia, and cirrhosis.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term “modulate” as used herein includes the inhibition or suppression of a function or activity (such as cell signaling) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

By “marker” is meant any protein or polynucleotide having an alteration in expression level or activity that is associated with a disease or disorder.

As used herein, “obtaining” as in “obtaining an agent” includes synthesizing, purchasing, or otherwise acquiring the agent.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disease, disorder, condition, and/or symptoms associated therewith. It will be appreciated that, although not precluded, treating a disease, disorder, or condition does not require that the disorder, condition or symptoms associated therewith be completely eliminated.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

Unless specifically stated or obvious from context, as used herein, the term “or” is understood to be inclusive. Unless specifically stated or obvious from context, as used herein, the terms “a,” “an,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

### Protein Deglycase DJ-1 (Dj1)

Dj1 (encoded by Park7 gene) is a small and highly conserved protein of 189 amino acids. Mutations in Dj1 cause autosomal recessive forms of Parkinson's disease (PD), and the protein has been implicated in various cellular processes, including homeostatic control of reactive oxygen species (ROS), transcription regulation, protein folding, modulation of glucose levels, fertility, and cellular transformation (3). Representative human nucleic and amino acid sequences of Dj1 are provided below.

```
Human Dj1 amino acid sequence (SEQ ID NO: 1):
>NP_001116849.1 Parkinson disease protein 7 [Homo sapiens]
    1 maskralvil akgaeemetv ipvdvmrrag ikvtvaglag kdpvqcsrdv vicpdasled
  61 akkegpydvv vlpggnlgaq nlsesaavke ilkeqenrkg liaaicagpt allaheigfg
121 skvtthplak dkmmngghyt ysenrvekdg liltsrgpgt sfefalaive alngkevaaq
181 vkaplvlkd
Human Dj1 nucleic acid sequence (mRNA, CDS 49-618) (SEQ ID NO: 2)
>NM_001123377.2 Homo sapiens Parkinsonism associated deglycase (PARK7) ,
transcript variant 2, mRNA
      1 gcgttcattt tcagcctggt gtggggcttg taaacatata acataaaaat ggcttccaaa
    61 agagctctgg tcatcctggc taaaggagca gaggaaatgg agacggtcat ccctgtagat
  121 gtcatgaggc gagctgggat taaggtcacc gttgcaggcc tggctggaaa agacccagta
  181 cagtgtagcc gtgatgtggt catttgtcct gatgccagcc ttgaagatgc aaaaaaagag
  241 ggaccatatg atgtggtggt tctaccagga ggtaatctgg gcgcacagaa tttatctgag
  301 tctgctgctg tgaaggagat actgaaggag caggaaaacc ggaagggcct gatagccgcc
  361 atctgtgcag gtcctactgc tctgttggct catgaaatag gttttggaag taaagttaca
  421 acacaccctc ttgctaaaga caaaatgatg aatggaggtc attacaccta ctctgagaat
  481 cgtgtggaaa aagacggcct gattcttaca agccgggggc ctgggaccag cttcgagttt
  541 gcgcttgcaa ttgttgaagc cctgaatggc aaggaggtgg cggctcaagt gaaggctcca
  601 cttgttctta aagactagag cagcgaactg cgacgatcac ttagagaaac aggccgttag
  661 gaatccattc tcactgtgtt cgctctaaac aaaacagtgg taggttaatg tgttcagaag
  721 tcgctgtcct tactactttt gcggaagtat ggaagtcaca actacacaga gatttctcag
  781 cctacaaatt gtgtctatac atttctaagc cttgtttgca gaataaacag ggcatttagc
  841 aaactactga ttgtttcttg ttttgtctct catttctttt gtgaaattaa attccgtatc
  901 accttcattt gcagctctta actgtccata tggcactgaa ataaaagaac agtgaccaca
  961 ttttacacag caaggaggaa aggcatacaa acagaattta agaggcttgt gattttctct
1021 gcttattagc tgtgtgtttt taatgtgcta ttaaaaaata ccaatgagg
Mouse (We only tested tDj1 in mouse models) tDj1 amino acid sequence (SEQ ID NO: 3)
  32 kvtvaglag kdpvqcsrdv micpdtsled
  61 aktqgpydvv vlpggnlgaq nlsespmvke ilkeqesrkg liaaicagpt allahevgfg
121 ckvtthplak dkmmngshys ysesrvekdg liltsrgpgt sfefalaive
Mouse Dj1 Amino Acid Sequence (SEQ ID NO: 4)
>NP_065594.2 Parkinson disease protein 7 homolog [Mus musculus]
    1 maskralvil akgaeemetv ipvdvmrrag ikvtvaglag kdpvqcsrdv micpdtsled
  61 aktqgpydvv vlpggnlgaq nlsespmvke ilkeqesrkg liaaicagpt allahevgfg
121 ckvtthplak dkmmngshys ysesrvekdg liltsrgpgt sfefalaive alvgkdmanq
181 vkaplvlkd
Mouse Dj1 nucleic acid sequence (mRNA, CDS 156-725) (SEQ ID NO: 5)
>NM_020569.3 Mus musculus Parkinson disease (autosomal recessive, early
onset) 7 (Park7), mRNA
    1 tgacgcaggc cgaggcggaa gcgaggggtt gtgcgcagcc tcagtgcggg tggcgcgcat
  61 gcgtgctggg tacacgtcgg gtgcgaggtt cctgcggact agcggtggct tcgcgtgggt
121 ggaggaggcg cggctgcagt ctttaagaaa tagaaatggc ttccaaaaga gctctggtca
181 tcctggccaa aggagcagag gagatggaga cagtgattcc tgtggatgtc atgcggcgag
241 ccgggatcaa agtcactgtt gcaggcttgg ctgggaagga ccccgtgcag tgtagccgtg
301 atgtaatgat ttgtccagat accagtctgg aagatgcaaa aacgcaggga ccatacgatg
361 tggtggttct tccaggagga aatctgggtg cacagaattt atctgagtcg cctatggtga
421 aggagatcct caaggagcag gagagcagga agggcctcat agctgccatc tgtgcaggtc
481 ctacggctct gttggctcac gaagtaggtt ttggatgcaa ggtcacaaca cacccactgg
541 ctaaggacaa aatgatgaat ggcagtcact acagctacte agagagccgc gtggagaagg
601 acggcctgat cctcaccagc cgcgggccgg ggaccagctt tgagtttgca ctagccattg
661 tggaggcact cgtggggaaa gacatggcca accaagtgaa ggcaccgctt gttctcaaag
721 actagagccc aagccctggg ccccacgctt gagcaggcat tggaagccca ctggtgtgtc
781 cagagcccag ggaacctcag cagtagtatg tgaagcagcc gccacacggg gctctcatcc
841 cgggtctgta tgtttctgaa ccttgctagt agaataaaca gtttaccaag ctcctgccag
901 ctaaaaaaaa aaaaaaaaaa
```

It is widely accepted that Dj1 protects neuronal cells by directly quenching reactive oxygen species (ROS) upon oxidative modification of a conserved cysteine residue and promotes efficient fuel utilization in other organs such as skeletal muscle (4). Moreover, numerous studies have found that Dj1 level was elevated in the body fluid such as plasma, urine, and cerebrospinal fluid in Parkinson's disease (PD) patients (5-11). However, the molecular mechanisms underlying circulating Dj1 function remain unclear. This disclosure provides novel compositions and methods based on the discoveries presented herein related to Dj1 expression and function.

For example, nucleic acid molecules encompassed by the present invention encode a Dj1 polypeptide or a fragment thereof. If the polynucleotide is a fragment of the full-length coding sequence of a Dj1 polypeptide, then the polynucleotide encodes a biologically active fragment of a Dj1 polypeptide. Such Dj1 polynucleotides have a nucleotide sequence that is at least about 50%, at least about 60%, at least about 70%, at least about 80%, at least about 90%, or at least about 95% or more (e.g., about 98%) homologous to the nucleotide sequence shown in SEQ ID NO: 2 or 5 or a portion thereof (i.e., 100, 200, 300, 400, 450, 500, or more nucleotides). Such polynucleotides may be isolated using standard molecular biology techniques and the sequence information provided herein. Alternatively, such polynucleotides may be isolated by polymerase chain reaction using oligonucleotide primers designed based upon the sequence of SEQ ID NO: 2 or 5, or fragment thereof, or the homologous nucleotide sequence. For example, mRNA may be isolated from cells (i.e., by the guanidinium-thiocyanate extraction procedure of Chirgwin et al. (1979) Biochemistry 18:5294-5299) and cDNA may be prepared using reverse transcriptase (i.e., Moloney MLV reverse transcriptase, available from Gibco/BRL, Bethesda, MD; or AMV reverse transcriptase, available from Seikagaku America, Inc., St. Petersburg, FL). Synthetic oligonucleotide primers for PCR amplification may be designed based upon the nucleotide sequence of SEQ ID NO: 2 or 5, or fragment thereof, or to a homologous nucleotide sequence. A nucleic acid encompassed by the present invention can be amplified using cDNA or, alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to a Dj1 nucleotide sequence may be prepared by standard synthetic techniques, i.e., using an automated DNA synthesizer.

Nucleic acid molecules encoding other Dj1 members that have a nucleotide sequence differing from SEQ ID NO: 2 or 5, or fragment thereof, are contemplated. Moreover, nucleic acid molecules encoding Dj1 proteins from different species (e.g., mouse), and thus have a nucleotide sequence that differs from the Dj1 sequence of SEQ ID NO: 2 or 5, are also intended to be within the scope of the present invention.

In certain embodiments, nucleic acid molecule(s) encompassed by the present invention encode a protein or portion thereof that includes an amino acid sequence sufficiently homologous to an amino acid sequence of SEQ ID NO: 1, 3, or 4, or fragment thereof, such that the protein or portion thereof increases fatty acid oxidation and decreases lipid content in the liver by improving mitochondrial activity, suppresses ASK1-JNK-PPARα signaling in the liver, improves glucose homeostasis in non-alcoholic steatohepatitis (NASH) mice, reduces inflammation and cell apoptosis in the liver, and ameliorates liver fibrosis.

As used herein, the language “sufficiently homologous” refers to proteins or portions thereof that have amino acid sequences that include a minimum number of identical or equivalent amino acid residues (e.g., an amino acid residue that has a similar side chain as an amino acid residue in SEQ ID NO: 1, 3, or 4, or fragment thereof) to an amino acid sequence of SEQ ID NO: 1, 3, or 4, or fragment thereof, such that the protein or portion thereof, once administered to a subject 1) increases fatty acid oxidation; 2) decreases lipid content in the liver; 3) suppresses ASK1-JNK-PPARα signaling in the liver; 4) improves glucose homeostasis in non-alcoholic steatohepatitis (NASH) mice; and 5) reduces inflammation, cell apoptosis, and fibrosis in the liver.

The Dj1 protein, or fragment thereof, can be at least about 50%, 70%, 75%, 80%, 85%, 90%, 91%, 92%, 93%, 94%, 95%, 96%, 97%, 98%, 99% or more homologous to the entire amino acid sequence of SEQ ID NO: 1, 3, or 4 or fragment thereof. The proteins of the present invention have at least one biologically active portion. As used herein, the term “biologically active portion” is intended to include a portion, e.g., a domain/motif, of Dj1 that has one or more of the biological activities of the full-length Dj1 protein, such as listed above.

The polynucleotides and polypeptides described herein can be isolated or purified using methods well known in the art. The Dj1 polynucleotides and polypeptides can be recombinant nucleic acid molecules or polypeptides. Dj1 polynucleotides can be DNA or RNA. In some embodiments, the Dj1 polynucleotides are incorporated into a vector (e.g., a cloning or expression vector).

Dj1 agonists are also contemplated herein. Relevant agonists increase the expression or activity of a Dj1 polynucleotide or polypeptide. An agonist may directly increase the expression or activity of a Dj1 polynucleotide or polypeptide or the agonist may act indirectly. For example, an agonist may act indirectly to increase the expression or activity of a Dj1 polynucleotide or polypeptide by promoting the activity or expression of a polynucleotide or polypeptide that acts to increase the expression or activity of a Dj1 polynucleotide or polypeptide. Alternatively, a Dj1 agonist may act indirectly by inhibiting the expression of activity of a polynucleotide or polypeptide that, when expressed, inhibits, impairs, or otherwise downregulates the expression or activity of a Dj1 polynucleotide or polypeptide. For example, an inhibitory nucleic acid (e.g., an siRNA) can inhibit expression of activity of a polynucleotide that, when expressed, inhibits, impairs, or otherwise downregulates the expression or activity of a Dj1 polynucleotide or polypeptide. Dj1 agonists are known in the art (e.g., sodium phenylbutyrate and D2R) have been shown to upregulate the expression or activity of Dj1 (53).

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition, nucleic acid, or protein is preferably administered as a pharmaceutical composition comprising, for example, a protein of the invention and a pharmaceutically acceptable carrier. A composition can comprise, for example, a Dj1 polypeptide or a fragment thereof or a polynucleotide encoding a Dj1 polypeptide or a fragment thereof and a pharmaceutically acceptable carrier.

Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection that circumvents transport or diffusion through an epithelial barrier), the aqueous solution is pyrogen-free, or substantially pyrogen-free. The excipients can be chosen, for example, to effect delayed release of an agent or to selectively target one or more cells, tissues, or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutically acceptable carrier can contain physiologically acceptable agents that act, for example, to stabilize, increase solubility or to increase the absorption of a compound such as tofacitinib. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound that produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active Dj1 protein or fragment thereof or nucleic acid encoding such protein or fragment thereof with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary, or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

As used herein, the term “administering” a substance, such as a therapeutic entity to an animal or cell” is intended to refer to dispensing, delivering or applying the substance to the intended target. In terms of the therapeutic agent, the term “administering” is intended to refer to contacting or dispensing, delivering or applying the therapeutic agent to an animal by any suitable route for delivery of the therapeutic agent to the desired location in the animal, including delivery by oral, transdermal or parenteral administration. Administration by injection can include without limitation, intravenous, intramuscular, intraarterial, intradermal, intraperitoneal, subcutaneous, injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

The subject receiving this treatment is any animal in need, including primates, in particular humans, and animal models.

In certain embodiments, compounds of the invention may be used alone or conjointly administered with another type of therapeutic agent.

### Methods of Treatment

The present invention provides methods of treating disease and/or disorders or symptoms thereof that comprise administering a therapeutically effective amount of a pharmaceutical composition comprising a Dj1 protein or fragment thereof or a polynucleotide encoding a Dj1 polypeptide or a fragment thereof to a subject (e.g., a mammal such as a human). Thus, certain embodiments relate to a method of treating a subject suffering from or susceptible to a disease or disorder or symptom thereof. The method includes the step of administering to the subject a therapeutic amount of a Dj1 protein or fragment thereof or a polynucleotide encoding a Dj1 polypeptide or a fragment thereof sufficient to treat the disease or disorder or symptom thereof under conditions such that the disease or disorder is treated.

Identifying a subject in need of such treatment can be in the judgment of a subject or a health care professional and can be subjective (e.g., opinion) or objective (e.g., measurable by a test or diagnostic method).

The administration of a Dj1 protein or fragment thereof or a polynucleotide encoding a Dj1 polypeptide or a fragment thereof to a subject increases the expression or activity of a Dj1 protein. Increased expression or activity of a Dj1 protein or fragment thereof 1) increases fatty acid oxidation; 2) decreases lipid content in the liver; 3) suppresses the ASK1-JNK signaling in the liver; 4) improves glucose homeostasis in subjects that have non-alcoholic steatohepatitis (NASH); and 5) reduces inflammation, cell apoptosis, and fibrosis in the liver. Subjects having or suspected of having a metabolic disease or disorder, for example, may be characterized by impaired glucose homeostasis, increased lipid content in the liver, and other symptoms, which may be alleviated by administration of a Dj1 protein or fragment thereof. Examples of metabolic diseases or disorders include, but are not limited to, non-alcoholic fatty liver disease (NAFLD), steatohepatitis, type II diabetes, hyperglycemia, hyperlipidemia, dyslipidemia, obesity, hyperinsulinemia, insulin resistance, hypercholesterolemia, non-familial hypercholesterolemia, familial hypercholesterolemia, heterozygous familial hypercholesterolemia, homozygous familial hypercholesterolemia, mixed dyslipidemia, atherosclerosis, early onset coronary heart disease, dyslipidemia, hypertriglyceridemia, hyperfattyacidemia, and cirrhosis. Accordingly, the invention generally features methods of increasing or promoting the expression and/or activity of a Dj1 protein or fragment thereof in a subject having or at risk of developing a metabolic disorder. Therapies provided by the invention include polypeptide therapies and polynucleotide therapies. In one embodiment, the method involves contacting a liver cell of the subject with a Dj1 protein or fragment thereof or a polynucleotide encoding a Dj1 polypeptide or a fragment thereof.

In one aspect, methods of treating a metabolic disease or disorder or symptoms thereof are provided that comprise administering a therapeutically effective amount of a pharmaceutical composition comprising an agent described herein that increases expression or activity a Dj1 protein or fragment thereof to a subject (e.g., a mammal, such as a human). The method includes the step of administering to the mammal a therapeutic amount of an agent described herein sufficient to treat the disease or disorder or symptom thereof under conditions such that the disease, disorder, or symptom is treated.

Another aspect provides a method of reducing fat accumulation in the liver of a subject in which the subject is administered a therapeutically effective amount of a Dj1 polypeptide or fragment thereof or a polynucleotide encoding a Dj1 polypeptide or fragment thereof. In some embodiments, the subject being administered a Dj1 protein or fragment thereof or a polynucleotide encoding a Dj1 polypeptide or a fragment thereof is also administered a lipid-lowering agent. Examples of lipid lowering agents include atorvastatin, simvastatin, rosuvastatin, fluvastatin, ezetimibe, niacin, bezafibrate, ciprofibrate, clofibrate, gemfibrozil, and fenofibrate.

Dj1 polypeptides can reduce blood glucose levels and as such represent a novel approach to treating type 2 diabetes. Accordingly, a method is provided for blood glucose levels in which a subject is administered a therapeutically effective amount of a Dj1 polypeptide or fragment thereof or a polynucleotide encoding a Dj1 polypeptide or a fragment thereof. This method can further comprise administering to the subject a therapeutically effective amount of insulin, GLP-1, metformin, or a DPP4 inhibitor.

In certain embodiments, a Dj1 agonist may be used instead of, or conjointly with, a Dj1 polypeptide or polynucleotide in the therapeutic methods described herein.

Such treatments will be suitably administered to subjects, particularly humans, suffering from, having, susceptible to, or at risk for a disease, disorder, or symptom thereof. Administration of the therapeutic agent can be accomplished orally, transdermally, or parenterally. Administration by injection can include without limitation, intravenous, intramuscular, intraarterial, intradermal, intraperitoneal, and subcutaneous injection or infusion.

### Diagnostic and Prognostic Methods

As demonstrated herein, Dj1 can be released from muscle cells, and an increased expression level of Dj1 in a subject sample relative to a normal control is correlated with a muscle injury. Thus, Dj1 is a marker for muscle injury. Based on this discovery, methods are provided for detecting the existence of muscle injury, determining susceptibility to muscle injury in a subject, and determining the progression of muscle injury in a subject. Each of these methods comprises detecting or assessing the expression level of Dj1.

Marker expression can be detected and/or measured at the transcript or protein level by methods well-known in the art. Detecting expressed transcripts (e.g., PARK7 transcripts) in a sample generally involves contacting the sample with a nucleic acid molecule comprising a nucleotide sequence that is at least partially complementary to the nucleic acid sequence of the transcript to be measured under conditions suitable for hybridization. In some embodiments, the nucleic acid molecule is labeled to allow visualization of the molecule after contacting the sample. In some embodiments, the nucleic acid molecule is present on the surface of a substrate. In some embodiments, the substrate comprises a microarray. In some embodiments, the nucleic acid molecule, or a portion thereof, present in a sample is amplified (e.g., by PCR, RT-PCR, etc.), and the amplified product is quantified, thereby providing an estimate of the concentration of the marker in a sample.

Marker expression can also be detected and quantified, in some embodiments, at the protein level. Antibodies that specifically bind a marker, or any other method known in the art, can be used to monitor expression of a marker of interest. Detection of an alteration relative to a normal reference sample can be used as a diagnostic indicator of muscle injury. Similarly, detection of alterations or similarities relative to a reference sample derived from a subject having muscle injury can be used as a diagnostic indicator of muscle injury.

The methods described herein can be used to diagnose an individual or to confirm the results of another diagnostic method. Additionally, the methods described herein, or known in the art, can be use used with any other diagnostic method described herein for a more accurate diagnosis of the presence or severity of muscle injury.

In some embodiments, individual markers (i.e., Dj1) are used in combination with other identified markers, or with other markers known in the art that are associated with muscle injury or treatment thereof. Individual markers, or combinations thereof, are differentially expressed in a subject and, therefore, differentially present in samples from a subject having muscle injury and from a normal subject in whom muscle injury is undetectable. In some embodiments, the Dj1 protein or polynucleotide level can be compared to a Dj1 protein or polynucleotide level observed in a control afflicted with the disease, disorder, or condition the subject has or is suspected of having. Thus, levels observed in the subject that are similar or the same as levels observed in an afflicted control are indicative of the subject having the disease, disorder, or condition.

In one aspect, a method is provided for detecting muscle injury in a subject by assessing the level of a Dj1 polypeptide or polynucleotide, wherein an elevated Dj1 polypeptide or polynucleotide level relative to a normal control indicates that the subject has muscle injury. In another aspect, a method is provided for determining susceptibility to muscle injury by assessing the level of a Dj1 polypeptide or polynucleotide, wherein an elevated Dj1 polypeptide or polynucleotide level relative to a normal control indicates that the subject is susceptible to muscle injury. Thus, elevated Dj1 levels would be expected in such an individual due to higher basal level of muscle injury than in a subject not predisposed to muscle injury. The progression of muscle injury can also be determined by assessing the level of a Dj1 polypeptide or polynucleotide, wherein an elevated Dj1 polypeptide or polynucleotide level relative to a normal control indicates muscle damage progression.

Subjects that have or are susceptible to muscle injury may have a disease or condition that predisposes them to muscle injury. Examples of diseases that could predispose someone to muscle damage include, but are not limited to, amyotrophic lateral sclerosis, Charcot-Marie-Tooth disease, multiple sclerosis, muscular dystrophy, myasthenia gravis, myopathy, myositis, peripheral neuropathy, spinal muscular atrophy, cardiac myopathy, rhabdomyolysis, myasthenia gravis, fibrositis, cramp, and sarcopenia.

### EXAMPLES

The invention, now being generally described, will be more readily understood by reference to the following examples, which are included merely for purposes of illustration of certain aspects and embodiments of the present invention and are not intended to limit the invention.

### Example 1: Materials and Methods

### Animals

C57BL/10cSn-Dmd^{mdx}/J (001801, mdx) and C57BL/10ScSnJ (000476, WT) mice were ordered directly from The Jackson Laboratory. Different ages of mdx and WT mice were obtained. Animals were weaned 21 days after birth and had free access to food and water and were housed on 12 h light-dark cycle. C57BL/6J males were introduced to a normal diet (NC, Research Diets D15100601) or high-fat diet (HFD, Research Diets D12331), and maintained on a diet for the indicated time. The HFD diet derives 58% of its kilocalories (kcal) from fat (soybean and coconut oil), 25.5% from carbohydrates (sucrose and maltodextrin), and 16.4% from protein (casein). NASH model mice were fed for up to 28 weeks with the NASH diet (Envigo, TD.120528) and 42% fructose (Sigma, F0127) in the water (1,2). The NASH diet is composed of 405.36 g/Kg sucrose and 12.5 g/Kg cholesterol. Recombinant mouse Dj1 protein (Novus, NBP2-59523) or truncated mouse Dj1 (tDj1, GenScript) protein was intravenously injected every day for 5 days or every other day for 2 weeks. Skeletal muscle-specific Park7 knockout mice were generated by crossing Park7 flox mice with Myl1-cre mice (JAX, #024713). Mice were raised in microisolator cages with a 12 h: 12 h light: dark cycle. All procedures were performed in accordance with the Guide for Care and Use of Laboratory Animals of the National Institutes of Health, and were approved by the Animal Subjects Committee of the University of California, Los Angeles.

### Exercise Training

Acute Endurance Exercise: Experimental groups comprised no running/sedentary (SED), 45 minutes exercise (EX45), 90 minutes exercise (EX90), and 90 minutes exercise+three-hour rest (EX90+3 hr Rest). The moderate-intensity endurance exercise protocol consisted of treadmill running (5% grade) at 15 m/min for the allotted time. This exercise protocol has been shown to represent moderate-intensity exercise in mice (3,4). All mice were acclimated to the treadmill by running for 10 minutes at 5-10 m/min on two separate occasions during the 2-4 days prior to experimentation. Mice in the SED groups were fasted for approximately six hours before tissue harvest. Mice in the EX90 were fasted for approximately three hours prior to exercising. Similarly, all mice were given access to water during the fasting period. Tissues were removed immediately following exercise completion and snap-frozen in liquid nitrogen prior to storage in a −80° C. freezer and further analysis.

Endurance exercise training: Experimental groups consisted of: no voluntary wheel running (SED) or 30 days of in cage voluntary wheel running (TRN). All mice were individually housed for three days prior to the introduction of the Respironics® Mini Mitter® (Bend, Oregon) in cage running wheels and were given two days to acclimate to the wheels. Wheels in the cages of SED mice were locked throughout the duration of the experiment. Daily running measurements and weekly body weights were taken for 30 consecutive days. After 30 days, running wheels within TRN cages were locked, and all mice remained within their cages for 24 hours. Following the 24-hour period, mice were fasted for approximately six hours after which tissues were removed immediately and snap-frozen in liquid nitrogen prior to storage in a −80° C. freezer and further analysis. Tissue samples were taken approximately 30 hours after locking the running wheels.

Downhill running (DHR): Mice designated for downhill running exercises were first allowed 5 min to ambulate freely on a motorized treadmill set at −10° grade with no belt speed in order to become familiarized with the laboratory environment (5). Following this stationary stage, mice warmed up by running for 6 min at a −22° grade, in which belt speed increased from 5 to 13 m/min. Mice were then given a 3 min rest period before performing a 90-min downhill running protocol that consisted of ten stages at a −22° grade. Each stage began with a 1-min acceleration period (from 5 to 12 m/min), followed by 5 min of constant running at 12 m/min. To prevent exhaustion and ensure that the mice could finish the protocol, each stage was separated by a 2-min rest period. Mice were occasionally prodded with a cotton-tipped applicator to encourage continuous running; the electric shock was prohibited because mice generally responded to a gentle tap on the tail or hindquarters.

### Cell Culture, Reagents, and Transfections

Mouse myoblast cells (C2C12) were maintained in high glucose DMEM/high, 10% fetal bovine serum with penicillin/streptomycin. pRK5-Dj1-HA plasmid was purchased from Addgene (#29396). Seeded cells were cultured in 6-well plates for 24 hours prior to transfection. Transfections were performed using Lipofectamine 2000 and PLUS reagent according to manufacturer instructions (Invitrogen, Carlsbad, CA). Human HepG2 cells were cultured in DMEM/low media containing 10% fetal bovine serum with penicillin/streptomycin. Mouse AML12 cells were maintained in DMEM/F12, ITS supplement (Gibco, USA), 40 ng/ml dexamethasone, and 10% fetal bovine serum with penicillin/streptomycin. For in vitro experiments, HepG2 cells were treated with or without recombinant human Dj1 protein (Novus, NBC1-18334) in serum-free media containing 25 mM glucose, 200 μM palmitic acid, and 0.25% bovine serum albumin for an indicated time.

### Adenovirus Dj1

An adenovirus encoding mouse Dj1 was generated using the ViraPower Adenoviral Expression System (Invitrogen) according to manufacturer instructions. Briefly, the coding sequence of the mouse Dj1 gene with flanking BP sites was amplified from the Addgene plasmid #29396 using High Fidelity Pfx50 DNA polymerase (Invitrogen). Entry clones were obtained by recombination of the purified DNA fragment with the pDONR221 vector using Gateway BP clonase II. Mouse Dj1 insert was transferred from the entry clones into the pAd/CMV/V5-DEST vector (Invitrogen) using the Gateway LR Clonase II enzyme mix. The recombinant adenoviral purified plasmid was used to generate adenoviral particles according to the manufacturer's instructions (Invitrogen). Adenoviral titer was determined using the AdenoX Rapid Titer Kit (Clontech).

### Histopathology

Cryosections (10-μm thick transverse sections of frozen muscle cut in a cryostat (Leica) at −20° C.) were subjected to hematoxylin & eosin (H&E) staining (10, 54). Liver tissues were fixed in phosphate-buffered 10% formalin and embedded in paraffin wax. Sections were cut and stained with H&E.

### Mitochondrial DNA

Total DNA was extracted from cells using DNeasy Blood and Tissue kit (Qiagen, Valencia, CA). Expression levels of mtCO₃ for mtDNA and 18S for nuclear DNA were assessed by real-time qPCR. The ratio of mtDNA (mtCO₃) to nuDNA (18S) was used as an estimate for comparing mtDNA content between the genotypes.

### Immunoblot Analyses

Mouse tissue and cell samples were pulverized in liquid nitrogen and homogenized in RIPA lysis buffer containing complete EDTA-Free protease (Roche) and Phosphatase Inhibitor Cocktail 2 (Sigma-Aldrich, St. Louis, MO). All lysates were centrifuged, clarified, and resolved by SDS-PAGE. Samples were transferred to PVDF membranes and subsequently probed with the following antibodies for protein and phospho-protein detection: Dj1 (R&D systems, AF3668; Abcam, ab11251), LC3B (Novus, Saint Charles, MO), Chop (Cell Signaling Technology, #2895T), Phospho-eIF2α^{Ser51} (Cell Signaling Technology, #3398S) and total eIF2α (Cell Signaling Technology, #5324S). Atf6α (Abcam, ab37149), Xbp1s (Cell Signaling Technology, #12782), Phospho-JNK^{Thr183/Tyr185} (Cell Signaling Technology, #9255S), t-JNK (Cell Signaling Technology, #9252S), Phospho-p38^{Thr180/Tye182} (Cell Signaling Technology, #4511T), t-p38 (Cell Signaling Technology, #8690T), Phospho-ASK1^{Ser967} (Cell Signaling Technology, #3764S), t-ASK1 (Cell Signaling Technology, #8662S), Phospho-TAK1^{Thr184/187} (Cell Signaling Technology, #4531), t-TAK1 (Cell Signaling Technology, #4505), Phospho-AMPK^{Thr172} (Cell Signaling Technology, #2535), t-AMPK (Cell Signaling Technology, #5831), Phospho-ACC^{Ser79} (Cell Signaling Technology, #3661), t-ACC (Cell Signaling Technology, #3676), Tom20 (Santa Cruz Biotechnology, sc-136211), Dgat1 (Abcam, ab54037), Cpt1α (Abcam, ab128568), Pparα (Santa Cruz Biotechnology, sc-398394), Atg1 (Cell Signaling Technology, #5831), Cd36 (Proteintech, #18836-1-AP), p62 (Cell Signaling Technology, #5114), Phospho-FoxO1 (Cell Signaling Technology, #84192), t-FoxO1 (Cell Signaling Technology, #2880), Cleaved-CASP3 (Cell Signaling Technology, #9661), Cleaved-CASP8 (Cell Signaling Technology, #8592), Cleaved-CAPS9 (Cell Signaling Technology, #20750), PARP (Cell Signaling Technology, #9532), c-Flip (Cell Signaling Technology, #56343), α-SMA (Santa Cruz Biotechnology, sc-53142), b-Actin (Santa Cruz Biotechnology, sc-47778), GAPDH (ThermoFisher Scientific, #PA1-987). Densitometric analyses were performed using BioRad Quantity One image software (Bio-Rad, Hercules, CA).

### ELISA

Mouse serum Dj1 and CK were measured by mouse Park7/DJ-1 DuoSet ELISA (DY8136, R&D systems) and mouse CKMB/Creatine Kinase MB ELISA Kit (LS-F5745, LSBio) respectively. Mouse Serum AST and ALT were measured by AST activity assay kit (Sigma, MAK055) and ALT activity assay kit (Sigma, MAK052) respectively.

### NEFA, Cholesterol, and Triglyceride Assay

Plasma and liver NEFA, Cholesterol, and Triglyceride were analyzed by their respective kits (FUJIFILM Wako Diagnostics)

### Liver Lipids Extraction

About 80 mg of liver samples were homogenized and sonicated in PBS (pH 7.4) for lipid extraction. An internal standard mixture (ISTD) and CHCl3:methanol (2:1) were added to the samples before being vortexed, mixed, sonicated, and centrifuged. The supernatant containing the lipid was removed and dried, the lipids were resuspended in ethanol and then sonicated.

### Plasma Beta-Hydroxybutyrate

Plasma beta-Hydroxybutyrate was measured by Cayman beta-Hydroxybutyrate (Ketone Body) Colorimetric Assay kit (Cayman, 700190).

### Mouse Plasma Mass Spectrometry

Mouse plasma samples were subject to immunoaffinity removal of 7 highly abundant proteins using Seppro Mouse Spin Columns prior to protein digestion with trypsin. Thermo Scientific TMT10plex Isobaric Mass Tag Labeling Reagents were utilized to provide multiplex relative quantitation. Peptides were fractionated via high pH reversed-phase chromatography then injected onto a laser-pulled nanopore C18 column with 1.8 μm beads and resolved using a 3-hour gradient optimized on a hybrid quadrupole-Orbitrap mass spectrometer in dd-MS2 mode. The raw data were analyzed in Proteome Discoverer 2.2, which provided measurements of abundance for the identified peptides.

### Intravital Imaging of Dj1

Recombinant mouse Dj1 protein (NBP2-59523, Novus) were labeled with near-infrared dyes using IRDye 800CW protein labeling kit-Low MW (Li-COR Biosciences) as described in the manufacturer's instructions. 10 μg of labeled Dj1 protein was injected into wild-type C57BL/6J mouse through the tail vein. The mice were imaged at the indicated time using an IVIS Spectrum (PerkinElmer). For tissue analysis, recipient mice were euthanized by cervical dislocation, and tissues were then dissected and imaged. Fluorescence was detected using excitation and emission filters at 745 nm and 800 nm, respectively.

### Live-Cell Imaging.

C2C12 cells were plated in CELLview 4-compartment glass-bottom tissue culture dishes (Greiner Bio-One, 627870), PS, 35/10 mm. CellMask Plasma Membrane Stains (C10046, Invitrogen) 100 nM 10-N-nonyl acridine orange, 15 nM TMRE, 5 μM Rho123, and/or 200 nM MitoTracker Green; Invitrogen) were mixed with cell culture media and incubated with GFP-tagged Dj1 transfected cells 1-3 hrs prior to live-cell imaging with an alpha Plan-Apochromat 100X/1.46 Oil DIC M27 objective on a Zeiss LSM 880 with Airyscan. Before image analysis, raw .czi files were automatically processed into deconvoluted Airyscan images using Zen software. Time-lapse images were acquired at approximately 1 frame/second.

### Statistics

Values presented are expressed as means±SEM. Statistical analyses performed using Student's t-test when comparing two groups of samples or one-way analysis of variance (ANOVA) with Tukey's post hoc comparison for identification of significance within and between groups using PASW Statistics 18 statistical software or GraphPad Prism 9. Two-way ANOVA was used for comparisons between mice that were grouped based on genotype. Three-way mixed ANOVA was used to determine group differences over time. Two-tailed independent and dependent Student's t-tests were also used when appropriate. Significance is set a priori at P<0.05.

### Example 2: Skeletal Muscle Releases Dj1

Dj1 protein has been detected in the plasma, urine, and cerebrospinal fluid of PD patients (6) and some cancer patients (7). Here, Dj1 protein was also detected in the culture medium of both primary mouse myotubes and C2C12 myotubes (immortalized mouse muscle cells) (FIG. 1F). Following treatment with 5 or 10 μl of adenovirus-Dj1, Dj1 levels in the culture medium of C2C12 myocytes were elevated while cellular Dj1 levels remained unchanged, suggesting that muscle cells release excess Dj1 protein (FIG. 1G). To further validate muscle cell releases of Dj1, Dj1 was overexpressed in C2C12 myocytes by transient transfection of HA-tagged Dj1, resulting in detection of both endogenous Dj1 and HA-tagged Dj1 in the culture medium. Interestingly, mitochondrial depolarizer CCCP treatment elevated HA-Dj1 levels in the media, but did not alter the protein level of cleaved caspase 3, indicating the muscle cell releases of Dj1 was not due to apoptosis (FIGS. 1H, 1I).

To verify that skeletal muscle release of Dj1 in vivo, the basal plasma Dj1 levels in male wild-type C57BL/6J mice were determined where levels were slightly increased from 20 ng/ml to 40 ng/ml in 2 to 20 weeks old mice (FIG. 1J). Physical exercise is known to induce the secretion of many myokines. To determine whether exercise stimulates skeletal muscle release of Dj1, plasma Dj1 levels were examined in the mice following acute treadmill and chronic running wheel training. It was found that exercise training did not alter plasma Dj1 levels (FIG. 1K).

Since CCCP administration elevated Dj1 levels in the media of C2C12 myotubes, it was hypothesized that muscle injury or stress may trigger skeletal muscle release of Dj1. In order to further explore this, plasma Dj1 levels were examined in three different muscle injury mouse models—dystrophin deletion mdx mice, downhill running mice, and a myotoxin injury mice.

The mdx mouse is a commonly used model to study Duchenne muscular dystrophy (DMD), the most common and severe muscular dystrophy (8). mdx skeletal muscles exhibit active myofiber necrosis (9), and enter a phase of florid myonecrosis at 3 weeks (10). To exclude the impact of myofiber necrosis on protein release, the plasma Dj1 level of 2 and 4 wk old mdx mice were determined. Surprisingly, both 2 and 4 weeks old mdx mice have strikingly increased circulating Dj1 levels with unchanged protein levels of Dj1 in skeletal muscle. In contrast, the plasma CKMB level, an indirect marker of muscle damage, was only elevated in 4 weeks old mdx mice (FIGS. 1A, 1B, 1L, and 1M). These results indicate that the plasma Dj1 levels could be a potential early diagnostic biomarker of muscle dystrophy.

Unaccustomed downhill running is known to induce muscle damage (11). After 90 minutes of downhill running, the mice had significantly increased plasma Dj1 levels when compared to the sedentary group (SED) (FIG. 1C).

Cardiotoxin (CTX) induces skeletal muscle injury and regeneration. To further validate the effect of muscle injury on circulating Dj1, C57BL/6J mice were injected intramuscularly with CTX, and plasma Dj1 levels were measured 2 days post-administration. Consistently, an increase of Dj1in the plasma was observed in CTX-treated mice (FIGS. 1D-1E, 1N, and 1O).

Moreover, plasma Dj1 levels were significantly reduced in the skeletal muscle-specific Park7 knockout mice (mPark7^{KO}) (>2 folds down) (FIGS. 1P-1Q), suggesting that skeletal muscle is a primary source of circulating Dj1. Ultimately, both in vitro and in vivo results indicate that skeletal muscles release Dj1.

### Example 3: Endoplasmic Reticulum (ER) Stress Induces Muscle Cells Release of Dj1

Strenuous exercise increases the endoplasmic reticulum (ER) stress, which at an excessive level can lead to muscle damage (12). Accumulating evidence suggests that ER stress is involved in myokine induction (13). To determine the mechanisms that drive skeletal muscle releases of Dj1, ER stress markers were examined in skeletal muscles of the three different mouse models described above. Consistently, muscles were found to have significantly elevated the protein levels or gene expression of ER stress markers Chop and Atf6α in all three mouse models (FIGS. 2A-2D), indicating that ER stress might be a trigger of skeletal muscle release of Dj1. To further validate muscle cells release of Dj1, in vitro studies were assessed. Indeed, both in vitro and ex vivo studies confirmed that muscle cells release Dj1 after being treated with the ER stress inducer Thapsigargin (Tg) as well as CCCP (FIGS. 2E, 2F, and 2L).

Extracellular vesicles are cell-derived membranous structures including exosomes and microvesicles bodies, which are enriched with proteins, lipids, DNA, and RNA (14). To examine whether Dj1 is released through the extracellular vesicle system, Dj1 levels were determined in the culture media of C2C12 myotubes that were treated with or without CCCP, Tg, exosome generation inhibitor GW4869, or exocytosis inhibitor Exo1. Both GW4869 and Exo1 treatment significantly reduced the protein level of Dj1 in the media, indicating that muscle cells release Dj1 in extracellular vesicles (FIG. 2G-2H). To visualize the release of Dj1 from muscle cells, time-lapse imaging was performed on C2C12 myocytes transfected with Dj1-GFP. Robust budding of vesicles from C2C12 myocytes treated with Tg or CCCP was observed (FIG. 2I). Based upon the release pattern and the average 1 μm diameter of budding vesicles, it was hypothesized that Dj1 was released in shedding microvesicles. To further determine which types of extracellular vesicles were enriched with Dj1, microvesicle bodies (MVBs) and exosomes from C2C12 culture media and CTX-treated mouse plasma were separated by differential centrifugation. Dj1 was detected in both MVBs and exosomes of the culture medium and mouse plasma (FIGS. 2J, 2K). Altogether, these findings showed that ER stress-activated skeletal muscle cells to release Dj1 in extracellular vesicles.

### Example 4: Recombinant Dj1 Protein Primarily Targets the Liver

Myokines contribute to whole-body metabolism by directly signaling to distant organs (15-18). To determine which organs Dj1 targets, WT mice were tail-vein injected with a recombinant Dj1 (rDj1) protein labeled with the near-infrared fluorescent dye IRDye 800CW, and the fluorescent signals were tracked using an optical imaging system (FIGS. 3I, 3J). A robust fluorescent signal could be detected within 5 minutes post-injection. And most signals were accumulated in the liver, kidney, and urine at 4 hours post-injection (FIGS. 3A-3C, 3K). Considering that the reagents injected through tail-vein often nonspecifically target the liver, fluorescent-labeled rDj1 was used to determine whether Dj1 could bind to the plasma membrane of liver cells. rDj1 was labeled with Alex 488 and added into the medium of mouse liver cells (AML12 cells) at the indicated time. Fluorescent microscopy detected that rDj1 colocalized with the plasma membrane of AML12 cells (FIG. 3D).

Furthermore, rDj1 treatment elevated the phosphorylation of AMPK and ACC and enhanced the oxygen consumption rate/extracellular acidification rate (OCR/ECAR) and mitochondrial ATP production rate in human liver carcinoma HepG2 cells (FIGS. 3E-3G and 3L-3N), indicating that rDj1 enhanced mitochondrial oxidative phosphorylation. RNA sequencing analysis further confirmed upregulated AMPK signaling in rDj1-treated HepG2 cells (FIG. 3H). These results indicate that rDj1 primarily targets hepatocytes and activates AMPK signaling and mitochondrial activity.

### Example 5: RDj1 Administration Suppresses High-Fat Diet Feeding Induced Hepatic Steatosis

AMPK signaling is vital for lipid metabolism in the liver (19). It was determined that rDj1 significantly promoted fatty acid oxidation which may contribute to the reduced triacylglycerol (TG) levels and Oil Red O staining (stain neutral lipids) in HepG2 cells (FIGS. 4A-4C). These findings indicate that Dj1 could enhance lipid metabolism and ameliorate HFD-induced hepatic steatosis. rDj1 was tail-vein injected into mice fed with a chow diet (NC) or high-fat diet (HFD) daily for 5 days. Surprisingly, rDj1 administration significantly reduced plasma non-esterified fatty acids (NEFA) and cholesterol levels in NC-fed mice, while decreasing TGs levels in the liver of HFD-fed mice (FIGS. 4D-4I). Consistently, liver H&E staining displayed fewer lipids in rDj1-treated HFD-fed mice (FIGS. 4J and 4T). Although body weights and fasting blood glucose levels of HFD-fed mice were unchanged during the rDj1 administration, rDj1 indeed enhanced glucose homeostasis and insulin action, as evidenced by improving glucose tolerance tests and insulin tolerance tests, respectively (FIGS. 4K-4M, 4U-4Z).

Elevated AMPK signaling and reduced lipid content in the liver led to the hypothesis that rDj1 might activate the whole-body energy metabolism of mouse. Therefore, the metabolic rate was determined in HFD-fed mice by indirect calorimetry. Interestingly, rDj1 administration elevated oxygen consumption (VO₂) and energy expenditure (EE), while reducing respiratory exchange ratio (RER) without changing food intake or activity (FIGS. 4N-4Q, 4AA-4GG). Enhanced mitochondrial complex I and complex II activity in isolated liver mitochondria from rDj1-treated mice suggest that rDj1 enhances energy expenditure by increasing mitochondria activity (FIGS. 4R-4S).

Taken together, these results suggest that rDj1 enhances mitochondrial respiration and improves energy metabolism in mice to combat HFD feeding-induced hepatic steatosis.

### Example 6: Dj1 Functional Domain is Vital to Inhibit Hepatic Steatosis

Dj1 is a highly conserved protein that is ubiquitously expressed in most mammalian tissues. It belongs to the Dj1/ThiJ/PfpI family chaperones, and its expression is induced by oxidative stress (20,21). To determine whether the PfpI domain of Dj1 exerts a similar role as the full-length Dj1 protein (rDj1) in liver cells, truncated Dj1 (tDj1) comprising of only the PfpI domain was purified (FIG. 5O). HFD-fed mice were intravenous injected with rDj1 or tDj1 every other day for 14 days. Intriguingly, both rDj1 and tDj1 administration significantly reduced the level of TG in the liver (FIGS. 5A-5F). In addition, tDj1 also reduced TG levels, but not NEFA levels, in palmitate acid-treated AML12 cells (FIGS. 5M and 5N). Consistently, liver histology showed less lipid accumulation in the liver of both rDj1 and tDj1 administrated mice (FIGS. 5G-5J, 5P-Q). Both rDj1 and tDj1 treatment also improved liver healthy by reducing plasma AST and ALT levels (FIGS. 5K, 5L). In conclusion, the PfpI domain of Dj1 exerts the main role of Dj1 in inhibiting diet-induced hepatic steatosis.

### Example 7: TDj1 Administration Regulates the ASK1-JNK-PPPARα Signaling in the Liver

As described above, Dj1 administration stimulates AMPK phosphorylation in the liver cells. However, elevated AMPK phosphorylation in vivo was not observed (data not shown). Numerous studies have reported that the c-Jun N terminal kinase (JNK) is critical to the development of steatosis and chronic liver injury in NAFLD (22-25). Here, JNK (p46 subunit) phosphorylation was significantly reduced in the liver of mice treated with rDj1 or tDj1, while p38 phosphorylation was only decreased in the liver of tDj1-treated mice (FIGS. 6A-6B), suggesting that Dj1 targets the mitogen-activated protein kinases (MAPK) pathway in the liver cells. However, not all MAPK pathways were activated. For example, phosphorylation of p42 was not altered by tDj1 treatment (FIG. 6O). Both rDj1 and tDj1 suppressed TNFα and Anisomycin-induced JNK (p54 and p46) phosphorylation in human HepG2 and mouse AML12 liver cells, respectively (FIGS. 6C-6D).

A wide variety of mechanisms activate the JNK pathway. In vitro and in vivo studies showed that tDj1 reduced the phosphorylation of apoptosis signal-regulating kinase 1 (ASK1), but not the transforming growth factor β-activated kinase 1 (TAK1) (FIG. 6E, 6P). Moreover, ASK1 is activated by various stresses, including oxidative stress, ER stress, calcium overload, and receptor-mediated inflammatory signals such as TNFα and lipopolysaccharide (LPS) (26,27). rDj1 administration suppressed both baseline and TNFα-induced ROS levels in HepG2 cells, which may contribute to reducing liver inflammation (FIG. 6F). It was found that Dj1 effectively reduced liver inflammation gene expression and the protein level of cleaved caspase 3 (FIG. 6G, 6Q, 6R).

Gene analysis showed that rDj1 treatment reduced gene expression of de-novo lipogenesis and gluconeogenesis in the liver of NC-fed mice, which were blunted in HFD-fed mice (FIGS. 6H, 6I). Dj1 treatment increased both mRNA expression and protein levels of peroxisome proliferator-activated receptor alpha (PPARα) in the liver (FIGS. 6J-JK). Also, the transcriptional activity of PPARα was determined to be elevated by Dj1 administration in the liver of both NC- and HFD-fed mice (FIGS. 6L, 6M, 6S, 6T). Furthermore, Dj1 elevated plasma β-hydroxybutyrate (a ketone body) levels in NC-fed mice but not in HFD-fed mice (FIG. 6N, 6U). These findings suggest that Dj1 inhibits inflammation and liver cell death through the ASK1-JNK-PPARα axis in the liver.

### Example 8: TDj1 Administration Ameliorates Inflammation and Hepatic Fibrosis

Selonsertib (ASK1 inhibitor) has failed to reach the primary efficacy endpoint of fibrosis improvement. However, inhibition of ASK1 and its downstream effector JNK1 are promising strategies to reduce liver cell death and hepatic fibrosis in preclinical studies (28-31). Given Dj1 inhibits Ask1 phosphorylation in vitro and in vivo, the impact of tDj1 on hepatic fibrosis was tested on mice fed a non-alcoholic steatohepatitis (NASH) diet fed for 28 weeks. tDj1 administration significantly reduced the body weight and improved both glucose and insulin tolerance (FIGS. 7A-7C). Unlike in HFD-fed mice, tDj1 did not considerably reduce the TG levels in the liver of the NASH diet-fed mice but significantly suppressed the total cholesterol level in the liver (FIGS. 7D, 7H, and 7N-7R). More interestingly, tDj1 administration significantly reduced fibrosis scores (FIGS. 7E, 7J, 7K) and robustly reduced liver ALT activity but not AST activity (FIGS. 7I, 7S). Consistent with previous findings in the HFD-feeding model, tDj1 significantly reduced both inflammatory gene expression and liver fibrosis gene expression (FIGS. 7F, 7L). More strikingly, tDj1 suppressed hepatic apoptosis, as shown by the TUNEL assay and decreased protein levels of apoptosis markers, including cleaved caspase 3, cleaved caspase 8, cleaved caspase 9, and cleaved PARP protein (FIGS. 7G, 7M). Together, these findings indicate that tDj1 administration ameliorates glucose homeostasis and dramatically suppresses inflammation, cellular apoptosis, and fibrosis gene expression in the liver of NASH mice.

As an endocrine organ, skeletal muscle produces and secretes hundreds of myokines that allow for crosstalk between the muscle and many other organs (16). The in vitro and in vivo studies presented herein have identified Dj1 as a novel myokine. Like the concepts of oxidative distress and oxidative eustress, overloaded ER stress is detrimental, while a low dose of ER stress is thought to be necessary for adaptive improvement by exercise and for increasing the health span (13,32,33). Although acute treadmill exercise and chronic running wheel exercise did not elevate plasma Dj1 levels, it was determined that ER stress, especially Atf6α-Chop activation, could stimulate muscle cells to release Dj1. Furthermore, consistently elevated circulating Dj1 in three muscle injury mouse models indicates that muscle cells release of Dj1 might be a compensatory response to ER stress initiated by muscle injury.

Almost all cells are capable of secreting extracellular vesicles comprising MVBs and exosomes enriched with proteins, nucleic acids, and lipids (14,34). Time-lapse imaging indicated that muscle cells release Dj1 via shedding vesicles. However, due to the boundaries of imaging and centrifuge procedures, the possibility of Dj1 proteins existing in exosomes cannot be excluded.

Dj1 activated the phosphorylation of AMPK and ACC in HepG2 cells and mouse primary hepatocytes indicating that circulating Dj1 may regulate energy metabolism in hepatocytes. Although the receptors of Dj1 on the plasma membrane have not been identified, Dj1 treatment elevated mitochondrial activity and reduced TG content in liver cells. In vivo studies presented herein consistently showed that Dj1 administration reduced TG contents in the liver, ameliorated HFD-feeding induced hepatic steatosis, and improved glucose homeostasis and insulin sensitivity. Mitochondria regulate hepatic lipid metabolism and oxidative stress. Accumulating evidence indicates that hepatic mitochondria play a critical role in the development and pathogenesis of steatosis and NAFLD (35,36). Dj1 administration enhanced the activities of both mitochondrial complex I and complex II, which may be a key contributor to the elevated fatty acid oxidation rate and enhanced energy homeostasis in the liver of mice.

The function of the PfpI domain of Dj1, the evolutionarily conserved amino acid sequence of the Dj1 protein, is not clear (37). Both the full-length recombinant Dj1 protein (rDj1) and tDj1 (only containing the PfpI domain) exerted similar effects in ameliorating hepatosteatosis, indicating that the PfpI domain is a critical protein domain of Dj1 in regulating liver metabolism.

ASK1 has been reported to promote inflammation, apoptosis, and fibrosis in the pathogenesis of NASH (29,38). Although selonsertib (ASK1 inhibitor) fails to reach the primary efficacy endpoint of fibrosis improvement (39) in Phase III clinical trial, patients receiving selonsertib demonstrated improvements in the stage of fibrosis, progression to cirrhosis, liver stiffness, and liver fat content in a Phase II trial (28). JNK kinase is one of the key downstream targets of the ASK1 (32), and recent investigations have indicated that the overactivation of JNK is critical to the development of NAFLD, suggesting that JNK may be a novel therapeutic target in NAFLD management (22,25). In vitro and in vivo studies presented herein showed that Dj1 treatment suppressed the phosphorylation of both ASK1 and JNK but not TAK1, suggesting that Dj1 specifically inhibited the ASK1-JNK pathway in the liver. JNK represses the activity of PPARα, which regulates lipid metabolism in the liver. Gene analysis showed that Dj1 treatment reduced both de novo lipogenesis (DNL) and gluconeogenesis and elevated plasma-hydroxybutyrate in the liver of NC-fed mice indicating that Dj1 administration could enhance lipid oxidation in the liver. However, these changes in gene expressions were not observed in the liver of HFD-fed mice, suggesting that HFD feeding blunts these effects. Moreover, elevated PPARα transcriptional activity was observed in the liver of NC-fed mice treated with rDj1. These findings indicate that Dj1 may boost energy metabolism and suppress hepatic steatosis through the ASK1-JNK-PPARα axis in the liver.

PPARα activation, in combination with PPARβ/δ agonism, can reduce inflammation and reverse mild liver fibrosis. Indeed, Dj1 administration improved glucose homeostasis and insulin action, reduced inflammation, inhibited cell apoptosis, and suppressed fibrosis gene expression in the liver of the mouse NASH model. The findings suggest that Dj1 may ameliorate hepatic inflammation, apoptosis, and fibrosis through the ASK1-JNK-PPARα pathway. Altogether, these findings indicate that Dj1 is a potential therapeutic agent for treating NAFLD.

### REFERENCES

1. Cohen, J. C., Horton, J. D., and Hobbs, H. H. (2011) Human fatty liver disease: old questions and new insights. Science 332, 1519-1523.
2. Younossi, Z. M., Stepanova, M., Younossi, Y., Golabi, P., Mishra, A., Rafiq, N., and Henry, L. (2020) Epidemiology of chronic liver diseases in the USA in the past three decades. Gut 69, 564-568.
3. Younossi, Z. M., Blissett, D., Blissett, R., Henry, L., Stepanova, M., Younossi, Y., Racila, A., Hunt, S., and Beckerman, R. (2016) The economic and clinical burden of nonalcoholic fatty liver disease in the United States and Europe. Hepatology 64, 1577-1586.
4. Dufour, J. F., Caussy, C., and Loomba, R. (2020) Combination therapy for non-alcoholic steatohepatitis: rationale, opportunities and challenges. Gut 69, 1877-1884.
5. Zhang, L., Wang, J., Wang, J., Yang, B., He, Q., and Weng, Q. (2020) Role of DJ-1 in Immune and Inflammatory Diseases. Front Immunol 11, 994.
6. Shi, S. Y., Lu, S. Y., Sivasubramaniyam, T., Revelo, X. S., Cai, E. P., Luk, C. T., Schroer, S. A., Patel, P., Kim, R. H., Bombardier, E., Quadrilatero, J., Tupling, A. R., Mak, T. W., Winer, D. A., and Woo, M. (2015) DJ-1 links muscle ROS production with metabolic reprogramming and systemic energy homeostasis in mice. Nat Commun 6, 7415.
7. Zhao, Z. H., Chen, Z. T., Zhou, R. L., Zhang, X., Ye, Q. Y., and Wang, Y. Z. (2018) Increased DJ-1 and alpha-Synuclein in Plasma Neural-Derived Exosomes as Potential Markers for Parkinson's Disease. Front Aging Neurosci 10, 438.
8. Waragai, M., Nakai, M., Wei, J., Fujita, M., Mizuno, H., Ho, G., Masliah, E., Akatsu, H., Yokochi, F., and Hashimoto, M. (2007) Plasma levels of DJ-1 as a possible marker for progression of sporadic Parkinson's disease. Neurosci Lett 425, 18-22.
9. Lin, X., Cook, T. J., Zabetian, C. P., Leverenz, J. B., Peskind, E. R., Hu, S. C., Cain, K. C., Pan, C., Edgar, J. S., Goodlett, D. R., Racette, B. A., Checkoway, H., Montine, T. J., Shi, M., and Zhang, J. (2012) DJ-1 isoforms in whole blood as potential biomarkers of Parkinson disease. Sci Rep 2, 954.
10. Hong, Z., Shi, M., Chung, K. A., Quinn, J. F., Peskind, E. R., Galasko, D., Jankovic, J., Zabetian, C. P., Leverenz, J. B., Baird, G., Montine, T. J., Hancock, A. M., Hwang, H., Pan, C., Bradner, J., Kang, U. J., Jensen, P. H., and Zhang, J. (2010) DJ-1 and alpha-synuclein in human cerebrospinal fluid as biomarkers of Parkinson's disease. Brain 133, 713-726.
11. Jang, J., Jeong, S., Lee, S. I., Seol, W., Seo, H., Son, I., and Ho, D. H. (2018) Oxidized DJ-1 Levels in Urine Samples as a Putative Biomarker for Parkinson's Disease. Parkinsons Dis 2018, 1241757.
12. Oda, M., Makita, M., Iwaya, K., Akiyama, F., Kohno, N., Tsuchiya, B., Iwase, T., and Matsubara, O. (2012) High levels of DJ-1 protein in nipple fluid of patients with breast cancer. Cancer Sci 103, 1172-1176.
13. Pardo, M., Garcia, A., Thomas, B., Pineiro, A., Akoulitchev, A., Dwek, R. A., and Zitzmann, N. (2006) The characterization of the invasion phenotype of uveal melanoma tumour cells shows the presence of MUC18 and HMG-1 metastasis markers and leads to the identification of DJ-1 as a potential serum biomarker. Int J Cancer 119, 1014-1022.
14. DeFronzo, R. A., and Tripathy, D. (2009) Skeletal muscle insulin resistance is the primary defect in type 2 diabetes. Diabetes Care 32 Suppl 2, S157-163.
15. Severinsen, M. C. K., and Pedersen, B. K. (2020) Muscle-Organ Crosstalk: The Emerging Roles of Myokines. Endocr Rev 41.
16. Charlton, M., Krishnan, A., Viker, K., Sanderson, S., Cazanave, S., McConico, A., Masuoko, H., and Gores, G. (2011) Fast food diet mouse: novel small animal model of NASH with ballooning, progressive fibrosis, and high physiological fidelity to the human condition. Am J Physiol Gastrointest Liver Physiol 301, G825-834.
17. Sellmann, C., Priebs, J., Landmann, M., Degen, C., Engstler, A. J., Jin, C. J., Garttner, S., Spruss, A., Huber, O., and Bergheim, I. (2015) Diets rich in fructose, fat or fructose and fat alter intestinal barrier function and lead to the development of nonalcoholic fatty liver disease over time. J Nutr Biochem 26, 1183-1192.
18. Davis, J. M., Kohut, M. L., Colbert, L. H., Jackson, D. A., Ghaffar, A., and Mayer, E. P. (1997) Exercise, alveolar macrophage function, and susceptibility to respiratory infection. J Appl Physiol (1985) 83, 1461-1466.
19. Moore, T. M., Zhou, Z., Cohn, W., Norheim, F., Lin, A. J., Kalajian, N., Strumwasser, A. R., Cory, K., Whitney, K., Ho, T., Ho, T., Lee, J. L., Rucker, D. H., Shirihai, O., van der Bliek, A. M., Whitelegge, J. P., Seldin, M. M., Lusis, A. J., Lee, S., Drevon, C. A., Mahata, S. K., Turcotte, L. P., and Hevener, A. L. (2019) The impact of exercise on mitochondrial dynamics and the role of Drp1 in exercise performance and training adaptations in skeletal muscle. Mol Metab 21, 51-67.
20. Baumann, C. W., Kwak, D., Ferrington, D. A., and Thompson, L. V. (2018) Downhill exercise alters immunoproteasome content in mouse skeletal muscle. Cell Stress Chaperones 23, 507-517.
21. Bulfield, G., Siller, W. G., Wight, P. A., and Moore, K. J. (1984) X chromosome-linked muscular dystrophy (mdx) in the mouse. Proc Natl Acad Sci USA 81, 1189-1192.
22. Yucel, N., Chang, A. C., Day, J. W., Rosenthal, N., and Blau, H. M. (2018) Humanizing the mdx mouse model of DMD: the long and the short of it. NPJ Regen Med 3, 4.
23. Duddy, W., Duguez, S., Johnston, H., Cohen, T. V., Phadke, A., Gordish-Dressman, H., Nagaraju, K., Gnocchi, V., Low, S., and Partridge, T. (2015) Muscular dystrophy in the mdx mouse is a severe myopathy compounded by hypotrophy, hypertrophy and hyperplasia. Skelet Muscle 5, 16.
24. Park, K. S., and Lee, M. G. (2015) Effects of unaccustomed downhill running on muscle damage, oxidative stress, and leukocyte apoptosis. J Exerc Nutrition Biochem 19, 55-63.
25. Pedersen, B. K. (2019) Physical activity and muscle-brain crosstalk. Nat Rev Endocrinol 15, 383-392.
26. Ost, M., Coleman, V., Kasch, J., and Klaus, S. (2016) Regulation of myokine expression: Role of exercise and cellular stress. Free Radic Biol Med 98, 78-89.
27. van Niel, G., D'Angelo, G., and Raposo, G. (2018) Shedding light on the cell biology of extracellular vesicles. Nat Rev Mol Cell Biol 19, 213-228.
28. Pedersen, L., and Hojman, P. (2012) Muscle-to-organ cross talk mediated by myokines. Adipocyte 1, 164-167.
29. Thyfault, J. P., and Bergouignan, A. (2020) Exercise and metabolic health: beyond skeletal muscle. Diabetologia 63, 1464-1474.
30. Pedersen, B. K., and Febbraio, M. A. (2012) Muscles, exercise and obesity: skeletal muscle as a secretory organ. Nat Rev Endocrinol 8, 457-465.
31. Smith, B. K., Marcinko, K., Desjardins, E. M., Lally, J. S., Ford, R. J., and Steinberg, G. R. (2016) Treatment of nonalcoholic fatty liver disease: role of AMPK. Am J Physiol Endocrinol Metab 311, E730-E740.
32. Dodson, M. W., and Guo, M. (2007) Pink1, Parkin, DJ-1 and mitochondrial dysfunction in Parkinson's disease. Curr Opin Neurobiol 17, 331-337.
33. Moore, D. J., West, A. B., Dawson, V. L., and Dawson, T. M. (2005) Molecular pathophysiology of Parkinson's disease. Annu Rev Neurosci 28, 57-87.
34. Czaja, M. J. (2010) JNK regulation of hepatic manifestations of the metabolic syndrome. Trends Endocrinol Metab 21, 707-713.
35. Manieri, E., Folgueira, C., Rodriguez, M. E., Leiva-Vega, L., Esteban-Lafuente, L., Chen, C., Cubero, F. J., Barrett, T., Cavanagh-Kyros, J., Seruggia, D., Rosell, A., Sanchez-Cabo, F., Gomez, M. J., Monte, M. J., JJ, G. M., Davis, R. J., Mora, A., and Sabio, G. (2020) JNK-mediated disruption of bile acid homeostasis promotes intrahepatic cholangiocarcinoma. Proc Natl Acad Sci USA 117, 16492-16499.
36. Das, M., Sabio, G., Jiang, F., Rincon, M., Flavell, R. A., and Davis, R. J. (2009) Induction of hepatitis by JNK-mediated expression of TNF-alpha. Cell 136, 249-260
37. Sabio, G., Cavanagh-Kyros, J., Ko, H. J., Jung, D. Y., Gray, S., Jun, J. Y., Barrett, T., Mora, A., Kim, J. K., and Davis, R. J. (2009) Prevention of steatosis by hepatic JNK1. Cell Metab 10, 491-498.
38. Nishitoh, H., Saitoh, M., Mochida, Y., Takeda, K., Nakano, H., Rothe, M., Miyazono, K., and Ichijo, H. (1998) ASK1 is essential for JNK/SAPK activation by TRAF2. Mol Cell 2, 389-395
39. Matsuzawa, A., Saegusa, K., Noguchi, T., Sadamitsu, C., Nishitoh, H., Nagai, S., Koyasu, S., Matsumoto, K., Takeda, K., and Ichijo, H. (2005) ROS-dependent activation of the TRAF6-ASK1-p38 pathway is selectively required for TLR4-mediated innate immunity. Nat Immunol 6, 587-592.
40. Loomba, R., Lawitz, E., Mantry, P. S., Jayakumar, S., Caldwell, S. H., Arnold, H., Diehl, A. M., Djedjos, C. S., Han, L., Myers, R. P., Subramanian, G. M., McHutchison, J. G., Goodman, Z. D., Afdhal, N. H., Charlton, M. R., and Investigators, G.-U . . . (2018) The ASK1 inhibitor selonsertib in patients with nonalcoholic steatohepatitis: A randomized, phase 2 trial. Hepatology 67, 549-559.
41. Schuster-Gaul, S., Geisler, L. J., McGeough, M. D., Johnson, C. D., Zagorska, A., Li, L., Wree, A., Barry, V., Mikaelian, I., Jih, L. J., Papouchado, B. G., Budas, G., Hoffman, H. M., and Feldstein, A. E. (2020) ASK1 inhibition reduces cell death and hepatic fibrosis in an Nlrp3 mutant liver injury model. JCI Insight 5.
42. Win, S., Than, T. A., Zhang, J., Oo, C., Min, R. W. M., and Kaplowitz, N. (2018) New insights into the role and mechanism of c-Jun-N-terminal kinase signaling in the pathobiology of liver diseases. Hepatology 67, 2013-2024.
43. Koch, P., Gehringer, M., and Laufer, S. A. (2015) Inhibitors of c-Jun N-terminal kinases: an update. J Med Chem 58, 72-95.
44. Timblin, G. A., Tharp, K. M., Ford, B., Winchester, J. M., Wang, J., Zhu, S., Khan, R. I., Louie, S. K., Iavarone, A. T., Ten Hoeve, J., Nomura, D. K., Stahl, A., and Saijo, K. (2021) Mitohormesis reprogrammes macrophage metabolism to enforce tolerance. Nat Metab 3, 618-635.
45. Chevet, E., Hetz, C., and Samali, A. (2015) Endoplasmic reticulum stress-activated cell reprogramming in oncogenesis. Cancer Discov 5, 586-597.
46. Schorey, J. S., Cheng, Y., Singh, P. P., and Smith, V. L. (2015) Exosomes and other extracellular vesicles in host-pathogen interactions. EMBO Rep 16, 24-43.
47. Morris, E. M., Meers, G. M., Booth, F. W., Fritsche, K. L., Hardin, C. D., Thyfault, J. P., and Ibdah, J. A. (2012) PGC-1alpha overexpression results in increased hepatic fatty acid oxidation with reduced triacylglycerol accumulation and secretion. Am J Physiol Gastrointest Liver Physiol 303, G979-992.
48. Piccinin, E., Villani, G., and Moschetta, A. (2019) Metabolic aspects in NAFLD, NASH and hepatocellular carcinoma: the role of PGC1 coactivators. Nat Rev Gastroenterol Hepatol 16, 160-174.
49. Lee, S. J., Kim, S. J., Kim, I. K., Ko, J., Jeong, C. S., Kim, G. H., Park, C., Kang, S. O., Suh, P. G., Lee, H. S., and Cha, S. S. (2003) Crystal structures of human DJ-1 and Escherichia coli Hsp31, which share an evolutionarily conserved domain. J Biol Chem 278, 44552-44559
50. Challa, T. D., Wueest, S., Lucchini, F. C., Dedual, M., Modica, S., Borsigova, M., Wolfrum, C., Bluher, M., and Konrad, D. (2019) Liver ASK1 protects from non-alcoholic fatty liver disease and fibrosis. EMBO Mol Med 11, e10124.
51. Harrison, S. A., Wong, V. W., Okanoue, T., Bzowej, N., Vuppalanchi, R., Younes, Z., Kohli, A., Sarin, S., Caldwell, S. H., Alkhouri, N., Shiffman, M. L., Camargo, M., Li, G., Kersey, K., Jia, C., Zhu, Y., Djedjos, C. S., Subramanian, G. M., Myers, R. P., Gunn, N., Sheikh, A., Anstee, Q. M., Romero-Gomez, M., Trauner, M., Goodman, Z., Lawitz, E. J., Younossi, Z., Stellar, and Investigators, S.-. (2020) Selonsertib for patients with bridging fibrosis or compensated cirrhosis due to NASH: Results from randomized phase III STELLAR trials. J Hepatol 73, 26-39.
52. Piccoli, G. B., Capizzi, I., Vigotti, F. N., Leone, F., D'Alessandro, C., Giuffrida, D., Nazha, M., Roggero, S., Colombi, N., Mauro, G., Castelluccia, N., Cupisti, A., and Avagnina, P. (2016) Low protein diets in patients with chronic kidney disease: a bridge between mainstream and complementary-alternative medicines? BMC Nephrol 17, 76.
53. Cuevas et al., Hypertension. 2012; 59:446-452.
54. Herbst et al. (2017) Digital PCR Quantitation of Muscle Mitochondrial DNA: Age, Fiber Type, and Mutation-Induced Changes. J Gerontol A Biol Sci Med Sci. 72 (10): 1327-1333.

1. A method of treating a metabolic disorder, reducing fat accumulation in the liver, or reducing blood glucose levels, comprising administering to a patient in need thereof a therapeutically effective amount of a Dj1 polypeptide or fragment thereof or a nucleic acid molecule encoding a Dj1 polypeptide or fragment thereof.

2. The method of claim 1, wherein the method is a method of treating a metabolic disorder, and the metabolic disorder is selected from non-alcoholic fatty liver disease (NAFLD), steatohepatitis, type II diabetes, hyperglycemia, hyperlipidemia, dyslipidemia, obesity, hyperinsulinemia, insulin resistance, hypercholesterolemia, non-familial hypercholesterolemia, familial hypercholesterolemia, heterozygous familial hypercholesterolemia, homozygous familial hypercholesterolemia, mixed dyslipidemia, atherosclerosis, early onset coronary heart disease, dyslipidemia, hypertriglyceridemia, hyperfattyacidemia, and cirrhosis.

3. (canceled)

4. The method of claim 1, wherein the method is a method of reducing fat accumulation in the liver, and the Dj1 polypeptide or fragment thereof or the nucleic acid molecule encoding a Dj1 protein or fragment thereof is co-administered with a therapeutically effective amount of a lipid-lowering agent.

5. The method of claim 4, wherein the lipid-lowering agent is selected from atorvastatin, simvastatin, rosuvastatin, fluvastatin, ezetimibe, niacin, bezafibrate, ciprofibrate, clofibrate, gemfibrozil, and fenofibrate.

6. (canceled)

7. The method of claim 1, wherein the method is a method of reducing blood glucose levels, and the Dj1 polypeptide or fragment thereof or the nucleic acid molecule encoding the Dj1 protein or fragment thereof is co-administered with a therapeutically effective amount of insulin, GLP-1, metformin, or a DPP4 inhibitor.

8. The method of claim 1, wherein the Dj1 polypeptide comprises an amino acid sequence having at least 70% sequence identity to SEQ ID NO: 1, 3, or 4.

9. The method of claim 1, wherein the Dj1 polypeptide comprises amino acid residues 32-173 of SEQ ID NO: 1.

10. The method of claim 1, wherein the Dj1 polypeptide consists of an amino acid sequence having at least 70% sequence identity to SEQ ID NO: 3.

11. The method of claim 1, wherein the amino acid sequence of the Dj1 polypeptide is at least 85% identical to SEQ ID NO: 1, 3, or 4.

12. The method of claim 1, wherein the nucleic acid molecule encoding the Dj1 polypeptide comprises a nucleic acid sequence having 70% identity to the nucleic acid sequence of SEQ ID NO: 2 or 5.

13. The method of claim 1, wherein the Dj1 polypeptide or fragment thereof or the nucleic acid molecule encoding the Dj1 protein or fragment thereof is chemically synthesized or purified from recombinant sources.

14. A composition comprising:
a Dj1 polypeptide or fragment thereof or a nucleic acid molecule encoding the Dj1 protein or fragment thereof; and
a pharmaceutically acceptable carrier, wherein the Dj1 polypeptide or fragment thereof comprises an amino acid sequence at least 70% identical to SEQ ID NO: 1, 3, or 4; or
wherein the nucleic acid molecule encoding the Dj1 polypeptide or fragment thereof comprises a nucleic acid sequence that is at least 70% identical to SEQ ID NO: 2 or 5.

15. The composition of claim 14, wherein the Dj1 polypeptide has the amino acid sequence of SEQ ID NO: 3.

16. A method of detecting, determining susceptibility to, or determining the progression of muscle injury in a subject, the method comprising determining a Dj1 polynucleotide or polypeptide level in a sample from the subject, wherein an elevated or increased Dj1 polynucleotide or polypeptide level in the subject relative to a reference or control is indicative of, indicates the subject is susceptible to, or indicates the progression of muscle injury.

17. (canceled)

18. The method of claim 16, wherein the reference is a previously determined level of the Dj1 polynucleotide or polypeptide from a normal subject.

19. The method of claim 16, wherein the control is the Dj1 polynucleotide or polypeptide level in a normal subject that has not suffered muscle injury.

20. The method of claim 16, wherein the method is a method for determining susceptibility to a muscle injury in a subject, the method comprising determining at least a first Dj1 polynucleotide or polypeptide level in a first subject sample and a second Dj1 polynucleotide or polypeptide level in a second subject sample, wherein an elevated second Dj1 polynucleotide or polypeptide level relative to the first Dj1 polynucleotide or polypeptide level is suggestive of susceptibility to the muscle injury.

21. (canceled)

22. The method of claim 16, wherein the muscle injury is a symptom of a disease or condition selected from amyotrophic lateral sclerosis, Charcot-Marie-Tooth disease, multiple sclerosis, muscular dystrophy, myasthenia gravis, myopathy, myositis, peripheral neuropathy, spinal muscular atrophy, cardiac myopathy, rhabdomyolysis, myasthenia gravis, fibrositis, cramp, and sarcopenia.

23. The method of claim 16, wherein the Dj1 polypeptide level is a measure of serum protein concentration of the Dj1 polypeptide.

24. The method of claim 16, wherein the Dj1 polynucleotide level is a measure of PARK7 mRNA levels.
